(19) United States
(12) Patent Application Publication
(10) Pub. No.: US 2007/0026406 A1
(43) Pub. Date: Feb. 1, 2007

(54) Apparatus and method for classifying multi-dimensional biological data

(75) Inventors: Laurent El Ghaoui, Berkeley, CA (US); Georges Natsoulis, Kensington, CA (US)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(73) Assignee: ICONIX PHARMACEUTICALS, INC., Mountain View, CA (US)

(21) Appl. No.: 10/568,094

(22) PCT Filed: Aug. 13, 2004
(86) PCT No.: PCT/US04/26835

§ 371 Date: May 17, 2006

Related U.S. Application Data
(60) Provisional application No. 60/495,081, filed on Aug. 13, 2003.
(60) Provisional application No. 60/494,975, filed on Aug. 13, 2003.

(51) Int. Cl. C12Q 1/68; G06F 19/00

(52) U.S. Cl. 435/6; 702/20

(57) ABSTRACT

Apparatus and method for classifying multi-dimensional biological data are described. In some embodiments, a methodology for deriving a linear classification rule can be used for predicting a biological activity or a biological state. Advantageously, the methodology described herein facilitates obtaining robust and sparse classifiers that account for uncertainty involved in real-world experiments and improve computational efficiency and ease of interpretation of results.

### FIELD OF THE INVENTION

The invention relates to apparatus and methods for classifying multi-dimensional biological data.

### BACKGROUND OF THE INVENTION

Genomic sequence information is now available for various organisms. The function of genes can be studied using polynucleotide arrays, which can be used to obtain vast amounts of gene expression data by, for example, quantifying the amount of various mRNA transcripts produced by a biological sample. Gene expression data obtained using polynucleotide arrays are often associated with multiple dimensions. In some instances, the number of dimensions can correspond to the number of genes for which measurements are made, a number which is often in the thousands.

With the vast amounts of gene expression data, techniques are desirable for analysis and interpretation of the data. In particular, it is desirable to develop techniques to identify relationships in gene expression data. A significant challenge of dealing with multi-dimensional biological data obtained using polynucleotide arrays is developing classification techniques that can be used to predict a biological activity or a biological state. Such classification techniques would dramatically improve the ability to apply gene expression data for drug development as well as for medical diagnosis and treatments.

It is against this background that a need arose to develop the apparatus and method described herein.

### SUMMARY OF THE INVENTION

In one embodiment, the present invention provides a method of identifying a biological activity of a compound of interest. The method comprises providing a plurality of reference gene expression datasets. Each gene expression dataset of the plurality of gene expression datasets includes a set of gene expression levels and a set of gene expression intervals. The plurality of gene expression datasets includes a first plurality of gene expression datasets associated with a first class of compounds and a second plurality of gene expression datasets associated with a second class of compounds. The first class of compounds has a first biological activity, and the second class of compounds has a second biological activity. The method also includes deriving a linear classification rule based on the plurality of gene expression datasets. Deriviation of the classification may be carried out by minimizing a loss function derived from the classification rule. In one preferred embodiment, the type of loss function used in the method is selected from the group consisting of support vector machine, logistic regression, and minimax probability machine. The method further includes applying the linear classification rule to a test dataset of gene expression levels associated with the compound of interest to identify a biological activity of the compound of interest as one of the first biological activity and the second biological activity. In one preferred embodiment, this method is carried out wherein the reference gene expression dataset is a chemogenomic dataset based on in vivo compound treatments.

Another embodiment of the invention relates to a method of identifying a biological state of a biological sample. The method includes providing a plurality of gene expression datasets. Each gene expression dataset of the plurality of gene expression datasets includes a set of gene expression levels and a set of gene expression intervals. The plurality of gene expression datasets includes a first plurality of gene expression datasets associated with a first biological state and a second plurality of gene expression datasets associated with a second biological state. The method also includes deriving a linear classification rule based on the plurality of gene expression datasets. Deriviation of the classification may be carried out by minimizing a loss function derived from the classification rule. In one preferred embodiment, the type of loss function used in the method is selected from the group consisting of support vector machine, logistic regression, and minimax probability machine. The method further includes applying the linear classification rule to a set of gene expression levels associated with the biological sample to identify a biological state of the biological sample as one of the first biological state and the second biological state.

In another embodiment, the present invention provides a method for classifying a test gene expression dataset comprising: providing a reference gene expression dataset; deriving a linear classification rule by reducing the value of a loss function associated with said reference gene expression dataset; and applying said linear classification rule to a test gene expression dataset thereby determining the classification of the test gene expression dataset. In one preferred embodiment, this method is carried out wherein the reference gene expression dataset is a chemogenomic dataset based on in vivo compound treatments. In another preferred embodiment, the type of loss function used in the method is selected from the group consisting of support vector machine, logistic regression, and minimax probability machine.

In another embodiment, the invention provides a computer program product for classifying a test gene expression dataset comprising: computer code for querying a reference gene expression dataset; computer code for deriving a linear classification rule by reducing the value of a loss function associated with said reference gene expression dataset; computer code for applying said linear classification rule to a test gene expression dataset and thereby determining the classification of the test gene expression dataset; and computer code for outputting the test dataset classification to the user. In one preferred embodiment, the computer code product uses a loss function is selected from the group consisting of support vector machine, logistic regression, and minimax probability machine.

### BRIEF DESCRIPTION OF THE DRAWINGS

FIG. 1 illustrates an example of a set of gene expression matrices, according to an embodiment of the invention.

FIG. 2 illustrates gene expression data plotted in a multi-dimensional space, according to an embodiment of the invention.

FIG. 3 and FIG. 4 illustrate results of numerical experiments for a logistic regression loss function, according to an embodiment of the invention.

FIG. 5 and FIG. 6 illustrate results of numerical experiments for a support vector machine loss function, according to an embodiment of the invention.

### DETAILED DESCRIPTION OF THE INVENTION

Embodiments of the invention relate to classifying multi-dimensional biological data. In particular, embodiments of the invention relate to identifying relationships in multi-dimensional biological data and predicting a biological activity or a biological state based on the identified relationships. For example, some embodiments of the invention relate to analyzing gene expression data and extracting relevant information from the sea of data that typically results from genomic experiments.

Embodiments of the invention can be used to derive a classification rule (i.e. a classifier or signature) for multi-dimensional biological data. Once derived, the classification rule can be used to classify a multi-dimensional data vector to one of various categories. For example, the multi-dimensional data vector can correspond to a set of gene expression levels obtained by exposing a biological sample to a compound. In accordance with the classification rule, the set of gene expression levels can be classified to one of various categories, and each category can be associated with a class of compounds having a particular biological activity. By classifying the set of gene expression levels to a particular category, a biological activity of the compound can be predicted.

As another example, the multi-dimensional data vector can correspond to a set of gene expression levels obtained from a biological sample. In accordance with the classification rule, the set of gene expression levels can be classified to one of various categories, and each category can be associated with a particular biological state. By classifying the set of gene expression levels to a particular category, a biological state of the biological sample can be predicted. In particular, an occurrence of or a progression towards the biological state can be predicted (e.g. prediction of onset of a disease state).

Multi-dimensional biological data can be provided approximately in terms of intervals of confidence. Such intervals can be associated with a range of experimental results obtained from multiple measurements or with any other experimental uncertainty. As described herein, a classification rule can be derived under the assumption that multi-dimensional biological data can vary within particular intervals. In one preferred embodiment, a methodology is used wherein the value of a loss function is reduced over possible realizations of the multi-dimensional biological data within particular intervals. In some instances, this methodology may be referred to as a “worse-case” methodology and can involve minimizing a worse-case value of a loss function over possible realizations of the multi-dimensional biological data.

The methodology described herein can be applied to derive classifiers using a variety of loss functions, including, for example, loss functions arising in the context of support vector machines (SVM), logistic regression (LR), and minimax probability machines (MPM). Advantageously, the methodology can lead to problems that are amenable to efficient solutions using convex optimization methods. Significantly, the methodology provides robust and sparse classifiers that account for uncertainty involved in real-world experiments. Use of these sparse classifiers improve computational efficiency and eases interpretation of results.

Definitions

The following definitions apply to some of the elements described with regard to some embodiments of the invention. These definitions may likewise be expanded upon herein.

The term “set” refers to a collection of one or more elements. Elements of a set can also be referred to as members of the set. Elements of a set can be the same or different. In some instances, elements of a set can share one or more common characteristics.

The term “biological sample” refers to a biological system or a model of a biological system. In some instances, a biological sample is capable of responding to a stimulus. Typical biological samples include, for example, individual cells, collections of cells (e.g., cell cultures), tissues, organs, multi-cellular organisms, prokaryotic organisms, populations of multi-cellular or prokaryotic organisms, and the like. For example, a biological sample can include a eukaryotic cell. Suitable eukaryotic cells include cells obtained from, for example, humans, rats, mice, cows, sheeps, dogs, cats, chickens, pigs, goats, yeasts, plants, and the like.

The term “biological state” refers to a condition associated with a biological sample. In some instances, a biological state can refer to one of two different conditions (e.g., a normal or disease condition or a non-toxic or toxic condition) or one of a number of different conditions (e.g., one of various disease conditions associated with different tumor types). A biological state can refer to an “inherent” condition associated with a biological sample or a condition in which the biological sample is exposed to a stimulus.

The term “stimulus” refers to a perturbation that can be applied to a biological sample. In some instances, a stimulus is capable of affecting a biological sample in accordance with a biological activity of the stimulus. For example, a stimulus can affect a biological sample and can induce a change in biological state of the biological sample. Typical stimuli include, for example, compounds, environmental stresses, and the like. Typical compounds include, for example, small organic molecules, such as drugs or prospective pharmaceutical lead compounds. Typical compounds can also include, for example, toxins, pollutants, dyes, flavors, herbal preparations, environmental agents, proteins, nutrients, peptides, polynucleotides, heterologous genes (e.g., in expression systems), plasmids, polynucleotide analogs, peptide analogs, lipids, carbohydrates, infectious agents (e.g., viruses, bacteria, fungi, parasites, and phages), and the like.

As used herein, the term “test compound” refers to a compound of interest, and the term “control compound” refers to a compound that is used as a standard of comparison. A control compound can be used to contrast biological activities of a test compound and of the control compound. In some instances, a control compound does not share any primary biological activity with a test compound. For example, control compounds can include drugs that are used to treat diseases distinct from those treated using test compounds. Additional examples of control compounds include vehicles, known toxins, known inert compounds, and the like. Typical environmental stresses include, for example, starvation, hypoxia, temperature changes, and the like.

The term “class of compounds” refers to a set of compounds having a similar or identical biological activity. For example, a class of compounds can refer to a set of compounds that share a primary biological activity. In some instances, a class of compounds can also refer to a set of compounds that lack a particular biological activity, such as, for example, a particular primary biological activity. An example of a class of compounds is a set of compounds prescribed for hyperlipoproteinemia, including fenofibrate, clofibrate, and gemfibrozil. Another example of a class of compounds is a set of non-steroidal anti-inflammatory compounds, including aspirin, ibuprofen, and naproxen.

The term “biological activity” or “bioactivity” refers to the ability of a stimulus to affect a biological sample. For example, a biological activity can refer to the ability of a compound to modulate the effect of an enzyme, block a receptor, stimulate a receptor, modulate the expression level of one or more genes, or a combination thereof. In some instances, a biological activity can refer to the ability of a stimulus to produce a toxic effect in a biological sample. Stimuli have a similar or identical biological activity when they produce a similar or identical effect in a biological sample in vivo or in vitro. For example, fenofibrate, clofibrate, and gemfibrozil have similar biological activities, and all three compounds are prescribed for hyperlipoproteinemia. Similarly, aspirin, ibuprofen, and naproxen have similar biological activities as all three are known to be non-steroidal anti-inflammatory compounds.

The term “primary biological activity” or “primary bioactivity” refers to the most pronounced or intended effect of a stimulus. For example, the primary biological activity of an ACE inhibitor is the inhibition of angiotensin-converting enzymes (and the concomitant reduction of blood pressure), regardless of secondary biological activities or side effects.

The term “modulated” and its grammatical equivalents refer to a change to a measurable or detectable degree. In some instances, the degree of change can be measured relative to a threshold or baseline value. For example, the term “modulated” in connection with gene expression can refer to a change in the expression level of a gene in the form of an induction (e.g., up-regulated) or repression (e.g., down-regulated) relative to a threshold or baseline expression level of the gene.

The term “gene expression dataset” refers to data that indicate expression levels of a set of genes. A gene expression dataset can be associated with a particular biological activity or a particular biological state. In some instances, a gene expression dataset can indicate one or more genes that are affected by a stimulus. For example, a gene expression dataset can indicate that a specific subset of genes of a genome is modulated by exposure to a compound, or other perturbation to the organism. A “reference” gene expression dataset may include gene expression data obtained under known and controlled conditions of a biological state or activity. The reference dataset may then be used to classify and thereby determine the biological state or activity of “test” gene expression dataset for which the particular biological state or activity of interest is unknown.

A gene expression dataset can include a set of data values associated with a set of genes for which measurements are made. A data value included in a gene expression dataset can indicate an expression level of a gene in absolute or relative terms. In some instances, a data value included in a gene expression dataset can indicate a typical expression level of a gene, such as, for example, an average or mean expression level of the gene in response to a stimulus. Gene expression levels can be measured as the level of mRNA transcription or using any other quantitative or qualitative methods of measuring gene expression levels. Gene expression levels can also be measured by, for example, quantifying the amount of encoded protein that is produced using proteomic techniques. A variety of methods for detecting amounts of encoded proteins may be used, including, for example, Western blots, ELISA, mass spectrometry, and 2-D gel electrophoresis.

Gene expression levels can be weighted or scaled to normalize data and can be expressed as an absolute increase or decrease in gene expression levels, a relative change in gene expression levels (e.g., a percentage change), the degree of change relative to threshold or baseline gene expression levels, and the like. For example, a gene expression level can be expressed in terms of an absolute quantity of mRNA transcribed for a gene, as a ratio of mRNA transcribed in response to a stimulus relative to a threshold or baseline value, or the like.

Alternatively, or in conjunction, a data value included in a gene expression dataset can indicate an expression interval of a gene. A gene expression interval can indicate a range of variation of an expression level of a gene, such as, for example, a range of variation of the expression level of the gene in response to a stimulus. A gene expression interval can be associated with multiple measurements of an expression level of a gene or with any other experimental uncertainty. As with gene expression levels, gene expression intervals can be expressed in absolute or relative terms and can be subjected to various manipulations, such as, for example, data normalization.

The terms “classification rule” or “classifier” refer to a statistical test for classifying multi-dimensional data, e.g. in answer to a “classification question.” A classification rule can be derived to classify multi-dimensional biological data with respect to various categories. These categories can be associated with different biological activities or different biological states. For example, a set of gene expression levels can be obtained by exposing a biological sample to a compound, and the set of gene expression levels can be classified to one of various categories. By classifying the set of gene expression levels to a particular category, a biological activity of the compound can be predicted. A classification rule can be a binary classification rule (i.e., for classifying multi-dimensional data into one of two categories) or a multi-class classification rule (i.e., for classifying multi-dimensional data into one of three or more categories).

A “classification question” may be of any type of question susceptible to yielding a yes or no answer (e.g. “Is the unknown a member of the class or does it belong with everything else outside the class?”).

A classification rule may be a linear classification rule (i.e., associated with a linear classification function) or a non-linear classification rule (i.e., associated with a non-linear classification function).

“Linear classifiers” refers to classifiers comprising a first order function of a set of variables, for example, a summation of a weighted set of gene expression logratios. A valid classifier typically is defined as a classifier capable of achieving a performance for its classification task at or above a selected threshold value. For example, a log odds ratio ≧4.00 represents a preferred threshold of the present invention. Higher or lower threshold values may be selected depending of the specific classification task.

“Log odds ratio” or “LOR” is used herein to summarize the performance of classifiers or signatures. LOR is defined generally as the natural log of the ratio of the odds of predicting a subject to be positive when it is positive, versus the odds of predicting a subject to be positive when it is negative. LOR is estimated herein using a set of training or test cross-validation partitions according to the following equation,

$$LOR = ln \frac{( {\sum }_{i = 1}^{c} {TP}_{i} + 0.5 ) * ( {\sum }_{i = 1}^{c} {TN}_{i} + 0.5 )}{( {\sum }_{i = 1}^{c} {FP}_{i} + 0.5 ) * ( {\sum }_{i = 1}^{c} {FN}_{i} + 0.5 )}$$

where c (typically c=40 as described herein) equals the number of partitions, and TPᵢ, TNᵢ, FPᵢ, and FNᵢ represent the number of true positive, true negative, false positive, and false negative occurrences in the test cases of the i^{th} partitition, respectively.

“Variable” as used herein, refers to any value that may vary. For example, variables may include relative or absolute amounts of biological molecules, such as mRNA or proteins, or other biological metabolites. Variables may also include dosing amounts of test compounds.

“Signature” as used herein, refers to a combination of variables, weighting factors, and other constants that provides a unique value or function capable of answering a classification question. A signature may include as few as one variable. Signatures include but are not limited to linear classifiers comprising sums of the product of gene expression logratios by weighting factors and a bias term.

“Weighting factor” as used herein, refers to a value used by an algorithm in combination with a variable in order to adjust the contribution of the variable.

“Impact factor” or “Impact” as used herein in the context of classifiers or signatures refers to the product of the weighting factor by the average value of the variable of interest. For example, where gene expression logratios are the variables, the product of the gene's weighting factor and the gene's measured expression logo₁₀ ratio yields the gene's impact.

The sum of the impacts of all of the variables (e.g. genes) in a set yields the “total impact” for that set.

“Scalar product” (or “Signature score”) as used herein refers to the sum of impacts for all genes in a signature less the bias for that signature. A positive scalar product for a sample indicates that it is positive for (i.e a member of) the classication that is determined by the classifier or signature.

The term “Group Signature” refers to data that can be used to identify a set of stimuli. In some instances, a Group Signature includes a group identifier and a set of gene identifiers. A group identifier can indicate a set of stimuli having a similar or identical biological activity (e.g., a class of compounds such as “fibrates”). Alternatively, or in conjunction, a group identifier can indicate a shared biological activity of a set of stimuli (e.g., PPARα activation) or the identity of stimuli belonging to the set of stimuli.

A gene identifier indicates a gene affected by a stimulus belonging to a set of stimuli. For example, gene identifiers can indicate which genes have expression levels that are modulated by exposure to a compound belonging to a set of compounds. Desirably, gene identifiers can indicate genes that are sufficiently characteristic or distinctive of a set of stimuli that modulation of expression levels of these genes in response to a stimulus can be used to identify whether the stimulus belongs to the set of stimuli. Also, the gene identifiers can be sufficiently characteristic or distinctive of the set of stimuli that the degree of modulation of expression levels of the genes in response to a stimulus can be used to identify whether the stimulus shares a biological activity with the set of stimulus. Gene identifiers can indicate genes by sequence, name, reference to an accession number, reference to a clone or position within a DNA array, and the like. Gene identifiers can further indicate gene expression levels for various genes in either absolute or relative terms. In particular, a gene identifier can indicate the direction or degree of gene expression modulation in absolute or relative terms. For example, a gene identifier can indicate that gene expression level is decreased by at least 10%, or that gene expression level is increased by between 100% and 500%. A gene identifier can further indicate time restrictions. For example, a gene identifier can indicate that gene 1 is up-regulated by at least 250% within 8 hours of administration or at not less than 4 hours but no more than 16 hours of administration.

Although a Group Signature may indicate any number of genes, it typically includes up to 200 gene identifiers of varying degrees of specificity from which subsets of varying specificity can be derived. Desirably, a Group Signature includes less than 50 gene identifiers, such as, for instance, less than 25 gene identifiers. For example, a Group Signature for a set of stimuli may include 20 gene identifiers. This Group Signature can include various sub-signatures having similar or lesser specificity, which sub-signatures can be derived by omitting one or more of the gene identifiers. In some instances, a Group Signature will include at least three gene identifiers, such as, for example, at least 5 gene identifiers, at least 10 gene identifiers, or at least 15 gene identifiers. In other instances, a Group Signature may include three or fewer gene identifiers. A Group Signature can further include bioassay data that can indicate, for example, a biological activity observed for a set of stimuli associated with the Group Signature. Bioassay data can be used to identify potential members of a set of compounds prior to genomic experiments, particularly where a number of drug candidates are to be screened. Bioassay data is particularly useful for distinguishing between compounds having unrelated structures, but which induce similar modulation in gene expression levels.

Various Group Signatures associated with different sets of compounds can be stored physically or electronically. A number of formats can be used for storing Group Signatures, including, for instance, tabular formats, relational databases, and multi-dimensional databases. A tabular format is a common format and can be implemented as spreadsheets, such as, for example, Microsoft Excel® and Corel Quattro Pro® spreadsheets. A relational database typically includes a set of tables composed of columns and rows for organizing data included in the database. Typically, a relational database supports a set of operations (e.g., select, join, combine, and the like) to manipulate data stored in the database. Suitable relational databases include, for example, Oracle® (Oracle Inc., Redwood Shores, Calif.) and Sybase® (Sybase Systems, Emeryville, Calif.) databases. For example, a large relational database of chemogenomic datasets may be constructed as described in U.S. patent application Ser. No. 10/854,609 filed May 24, 2004 (titled “Interactive Correlation of Compound Information and Genomic Information”) which is hereby incorporated by reference for all purposes.

For certain diagnostic applications, various Group Signatures can be embodied in an array of gene probes or set of diagnostic reagents, in full or in part. For example, a probe array having a separate region of probes specific for each Group Signature.

The term “Drug Signature” refers to data that can be used to identify a particular stimulus. In some instances, a Drug Signature refers to data similar to a Group Signature but specific to a particular stimulus or a set of substantially identical stimuli, such as, for example, salts or esters of a particular compound. Gene identifiers of a Drug Signature can be selected to distinguish a particular stimulus from other stimuli with which it shares a biological activity. For instance, Drug Signatures can be used to distinguish various stimuli associated with a Group Signature, various stimuli associated with different Group Signatures, or various unrelated stimuli.

The term “similar” refers to a degree of correspondence between two elements being compared. In some instances, two elements can be considered to be similar based on determining whether the difference between the two elements is within a particular threshold or baseline value. For example, two genes can be considered to be similar if they exhibit sequence identity of more than a particular threshold or baseline value, such as, for example, a sequence identity of 20%. A number of methods and systems for evaluating the degree of similarity of polynucleotide sequences are available, including, for example, BLAST, FASTA, and the like. Additional methods and systems include those disclosed in U.S. Pat. No. 5,953,727 to Maslyn et al. and U.S. Pat. No. 5,706,498 to Fujiyama et al., the disclosures of which are incorporated herein by reference in their entireties. Two sets of gene expression levels can be considered to be similar based on, for example, the number of identical genes affected, the degree to which each gene is affected, and the like. Several different measures of similarity or methods of scoring similarity can be used. For example, one measure of similarity considers each gene that is induced or repressed relative to a threshold or baseline level and increases a similarity score for each gene in which both sets indicate induction of that gene or in which both sets indicate a repression of that gene. A similarity score can take into account, for each gene, the level of modulation associated with that gene relative to other available gene expression data. Such similarity score can reflect that a set of gene expression levels matches a reference set of gene expression levels on multiple genes and that the degree of modulation for each gene is large. In some instances, a similarity score may be referred to as a “specificity score,” as it measures how rare the match of a set of gene expression levels to a reference set of gene expression levels is relative to remaining gene expression data. Additional discussion regarding similarity scores can be found in the co-pending and co-owned patent application to Eynon et al., entitled “Signature Projection Score,” U.S. application Ser. No. 60/435,883, filed Dec. 20, 2002, the disclosure of which is incorporated herein by reference in its entirety. Other statistical methods are also applicable. Similarity between a set of gene expression levels and a Group or Drug signature can be determined using a variety of methods, such as, for example, using a similarity score as discussed above.

The terms “polynucleotide,” “oligonucleotide,” “nucleic acid,” and “nucleic acid molecule” refer to a polymeric form of nucleotides of any length, including, for example, ribonucleotides and deoxyribonucleotides. These terms can refer to triple-, double-, and single-stranded DNA as well as triple-, double-, and single-stranded RNA. The terms can refer to modified forms, such as by methylation and/or by capping, and unmodified forms of a polynucleotide. For example, the terms can refer to polydeoxyribonucleotides (e.g., containing 2-deoxy-D-ribose), polyribonucleotides (e.g., containing D-ribose), any other type of polynucleotide which is an N- or C-glycoside of a purine or pyrimidine base, and other polymers containing non-nucleotidic backbones, such as, for example, polyamide (e.g., peptide nucleic acids (“PNAs”)) and polymorpholino polymers (e.g., commercially available as Neugene from Anti-Virals, Inc., Corvallis, Oreg.). The terms can also refer to various synthetic sequence-specific nucleic acid polymers in which the polymers include nucleobases in a configuration that allows for base pairing and base stacking such as found in DNA and RNA.

The term “probe” refers to a structure including a polynucleotide having a nucleic acid sequence capable of hybridizing to a polynucleotide present in a target analyte. In some instances, a probe includes a polynucleotide that is at least partially complementary to a target polynucleotide to be detected. Typically, a probe is labeled so that its presence can be detected. Polynucleotides of probes may be composed of DNA, RNA, synthetic nucleotide analogs, or a combination thereof. Probes of dozens to several hundred bases long can be artificially synthesized using oligonucleotide synthesizing machines or can be derived from various types of DNA cloning techniques. A probe can be single-stranded or double-stranded. Probes are useful in the detection, identification, and isolation of particular gene sequences or fragments. It is contemplated that a probe can be labeled with a reporter molecule, so that the probe is detectable using a detection system, such as, for example, ELISA, EMIT, enzyme-based histochemical assays, fluorescence, radioactivity, luminescence, spin labeling, and the like.

“Array” as used herein, refers to a set of different biological molecules (e.g. polynucleotides, peptides, carbohydrates, etc.). An array may be immobilized in or on one or more solid substrates (e.g. glass slides, beads, or gels) or may be a collection of different molecules in solution. An array may include a plurality of biological polymers of a single class (e.g. polynucleotides) or a mixture of different classes of biopolymers (e.g. an array including both proteins and nucleic acids immobilized on a single substrate).

The term “hybridize” and its grammatical equivalents refer to the coupling of polynucleotides that are sufficiently complementary to form a complex via Watson-Crick base pairing. It will be appreciated that the hybridizing sequences need not be perfectly complementary to provide stable complexes. Furthermore, the ability of two polynucleotides to hybridize can be dependent on experimental conditions. For example, temperature and salt concentration can affect the percentage of complementary base pair matches required for hybrid duplexes to remain intact. Conditions that favor hybridization are referred to as being less “stringent” than conditions that require a greater degree of sequence complementarity to maintain a stable complex. In many situations, stable complexes will form where fewer than about 10% of bases are mismatches, ignoring loops of four or more nucleotides. Accordingly, as used herein, the term “hybridize” can refer to the formation of a stable complex between a polynucleotide and its “complement” under appropriate experimental conditions and where there is typically about 90% or greater homology.

General Methodoloy

Construction of Gene Expression Datasets

The present invention may be used to generate classifiers useful for analyzing gene expression datasets obtained from biological samples. A typical biological sample includes a eukaryotic cell, such as, for example, a mammalian cell. Eukaryotic cells can be tested in vivo or in vitro. In some instances, it is desirable to examine eukaryotic cells obtained from various tissue types, such as, for example, liver, kidney, bone marrow, spleen, and the like. In one preferred embodiment the biological samples are tissues from compound-treated animals. A detailed description of the construction of such an in vivo gene expression dataset is described in U.S. patent application Ser. No. 10/854,609 filed May 24, 2004 (titled “Interactive Correlation of Compound Information and Genomic Information”) which is hereby incorporated by reference for all purposes.

Each biological sample of a set of biological samples can be exposed to a particular stimulus, and gene expression levels of a set of genes can be measured to obtain a gene expression dataset associated with the stimulus. A gene expression dataset can indicate that one or more gene expression levels are modulated by a stimulus. Gene expression levels can be expressed quantitatively, qualitatively, or both. For example, a gene expression level can be expressed quantitatively based on the amount of mRNA produced or qualitatively based on whether the gene expression level is up-regulated or down-regulated. Gene expression levels can be subjected to one or more manipulations, including, for example, data normalization based on comparing data from different regions of an array to adjust for any systematic errors. Gene expression levels can be expressed in either absolute or relative terms. In some instances, gene expression levels are expressed in the form of a ratio or a logarithm of a ratio. For example, a gene expression level may be expressed as a ratio of an expression level of a gene in response to a stimulus relative to a threshold or baseline expression level of the gene. The threshold or baseline expression level can be, for example, an expression level of the gene absent the stimulus, a historical expression level of the gene, a pooled or averaged expression level of a number of genes, and the like. As another example, a gene expression level may be expressed relative to a “dynamic range” of a gene (e.g., a maximum range of variation of the gene observed historically).

A biological sample can be exposed to a stimulus under particular experimental conditions and can be examined at various time points. Examples of experimental conditions include time, temperature, subject animal species, subject animal gender, subject animal age, other treatment of subject animal (e.g., environmental stresses, prior or concurrent administration of other compounds, and time and manner of sacrifice), tissue or cell line from which gene expression data is derived, type of array and serial number, date of experiment, researcher who performed experiment, client for whom experiment was performed, and the like.

For certain applications, it is desirable to analyze the effects of various stimuli concurrently, particularly where the stimuli are related by biological activity or therapeutic effect. For example, a biological sample can be exposed to a set of stimuli (e.g., a set of compounds), and gene expression levels of a set of genes can be measured to obtain a gene expression dataset associated with the set of stimuli.

In some instances, gene expression datasets obtained from a set of biological samples can indicate gene expression intervals of a set of genes. A gene expression interval can indicate a range of variation of an expression level of a gene, such as, for example, a range of variation of an expression level of the gene in response to a stimulus. Various statistical measures can be used to characterize gene expression intervals, including, for example, standard deviations, interquartile ranges, and the like. A gene expression interval can be associated with multiple measurements of an expression level of a gene or any other experimental uncertainty. Multiple measurements of a gene expression level may be made under the same or different experimental conditions. Also, multiple measurements of a gene expression level may be made using one or more biological samples. For example, multiple measurements of an expression level of a gene may be made using mammalian cells obtained from different tissue types.

General Methods of Measuring Gene Expression Levels

Gene expression levels of biological samples can be measured during or subsequent to exposing the biological samples to various stimuli. Gene expression levels can be easured using various methods, including, for example, employing a panel of reporter cells in which each group of cells has a reporter gene operatively connected to a different selected regulatory region. Alternatively, or in conjunction, gene expression levels can be obtained using primary tissue isolates or cells or cell lines lacking reporter genes. Methods for measuring gene expression levels include, for example, direct hybridization of mrRNA with oligonucleotides or longer DNA fragments (e.g., cDNA or fragments of cloned genomic DNA that can be in a solution or bound to a solid phase), reverse transcription followed by detection of the resulting cDNA, Northern blot analysis, and the like.

Primers and probes for measuring gene expression levels can be derived from gene sequences and are readily synthesized by standard techniques, such as, for example, solid phase synthesis via phosphoramidite chemistry disclosed in U.S. Pat. Nos. 4,458,066 and 4,415,732; Beaucage et al. (1992) Tetrahedron 48:2223-2311; and Applied Biosystems User Bulletin No. 13 (Apr. 1, 1987), the disclosures of which are incorporated herein by reference in their entireties. Other chemical synthesis methods include, for example, a phosphotriester method disclosed in Narang et al., Meth. Enzymol. (1979) 68:90 and a phosphodiester method disclosed in Brown et al., Meth. Enzymol. (1979) 68:109, the disclosures of which are incorporated herein by reference in their entireties. Poly(A), poly(C), or other non-complementary nucleotide extensions may be incorporated into probes using these methods. Hexaethylene oxide extensions may be coupled to probes by standard methods, such as, for example, disclosed in Cload et al. (1991) J. Am. Chem. Soc. 113:6324-6326; U.S. Pat. No. 4,914,210 to Levenson et al.; Durand et al. (1990) Nucleic Acids Res. 18:6353-6359; and Horn et al. (1986) Tet. Lett. 27:4705-4708, the disclosures of which are incorporated herein by reference in their entireties.

While the length of primers and probes can vary, probe sequences are typically selected such that they have a lower melting temperature than primer sequences. Hence, the primer sequences are typically longer than the probe sequences. Typically, the primer sequences are in the range of between 10-75 nucleotides long, more typically in the range of 20-45 nucleotides. A typical probe is in the range of between 10-50 nucleotides long, such as 15-40 nucleotides, 18-30 nucleotides, or any other length between the stated ranges.

If one or more solid supports are used, a probe may be attached to the solid support in a variety of manners. For example, a probe may be attached to a solid support by attachment of a 3′ or 5′ terminal nucleotide of the probe to the solid support. Examples of types of solid supports for immobilization of a probe include controlled pore glass, glass plates, polystyrene, avidin-coated polystyrene beads, cellulose, nylon, acrylamide gel, and activated dextran.

Desirably, a probe is attached to a solid support by a linker, which serves to distance the probe from the solid support. The linker is usually at least 15-30 atoms in length, typically at least 15-50 atoms in length. The required length of the linker can depend on the particular solid support used. For example, a six atom linker is generally sufficient when a highly cross-linked polystyrene is used as a solid support.

A wide variety of conventional linkers may be used to attach a probe to a solid support. A linker may be formed of any compound that does not significantly interfere with hybridization of a target sequence to a probe attached to a solid support. A linker may be formed of a homopolymeric oligonucleotide that can be readily added to the linker by automated synthesis. Polymers such as functionalized polyethylene glycol can also be used as linkers. Such polymers are sometimes particularly desirable because they do not significantly interfere with hybridization of a target sequence to a probe. Linkages between a solid support, a linker, and a probe are desirably not cleaved during removal of base protecting groups under basic conditions at high temperature. Examples of linkages include carbamate and amide linkages.

In some instances, probes may be coupled to labels to allow detection. Examples of labels include various molecules used for detection, including radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin, or haptens), and the like. The term “fluorescer” refers to a substance or a portion thereof that is capable of exhibiting fluorescence in a detectable range. Various methods for derivatizing oligonucleotides with reactive finctionalities can be used to add a label. In particular, several approaches are available for biotinylating probes such that radioactive, fluorescent, chemiluminescent, enzymatic, or electron dense labels can be attached via avidin. For example, labels can be added using methods disclosed in Broken et al., Nucl. Acids Res. (1978) 5:363-384, which discloses use of ferritin-avidin-biotin labels, and Chollet et al. Nucl. Acids Res. (1985) 13:1529-1541, which discloses biotinylation of the 5′ termini of oligonucleotides via an aminoalkylphosphoramide linker arm. Several methods are also available for synthesizing amino-derivatized oligonucleotides that are readily labeled by fluorescent or other types of compounds derivatized by amino-reactive groups, such as isothiocyanate, N-hydroxysuccinimide, and the like. Such methods include those disclosed in Connolly (1987) Nucl. Acids Res. 15:3131-3139; Gibson et al. (1987) Nucl. Acids Res. 15:6455-6467; and U.S. Pat. No. 4,605,735 to Miyoshi et al. Methods are also available for synthesizing sulffiydryl-derivatized oligonucleotides that can be reacted with thiol-specific labels, as, for example, disclosed in U.S. Pat. No. 4,757,141 to Fung et al.; Connolly et al. (1985) Nucl. Acids Res. 13:4485-4502; and Spoat et al. (1987) Nucl. Acids Res. 15:4837-4848. A comprehensive review of methodologies for labeling DNA fragments is provided in Matthews et al., Anal. Biochem. (1988) 169:1-25.

Probes may be fluorescently labeled by coupling a fluorescer to a non-ligating terminus of the probes. Guidance for selecting appropriate fluorescers can be found in Smith et al., Meth. Enzymol. (1987) 155:260-301; Karger et al., Nucl. Acids Res. (1991) 19:4955-4962; and Haugland (1989) Handbook of Fluorescent Probes and Research Chemicals (Molecular Probes, Inc., Eugene, Oreg.). Typical fluorescers include fluorescein and derivatives thereof, such as, for example, disclosed in U.S. Pat. No. 4,318,846 and Lee et al., Cytometry (1989) 10:151-164. Typical fluorescers also include 6-FAM, JOE, TAMRA, ROX, HEX-1, HEX-2, ZOE, TET-1, NAN-2, and the like.

Additionally, probes can be labeled with an acridinium ester (“AE”) label using various techniques, such as, for example, disclosed in Nelson et al. (1995) “Detection of Acridinium Esters by Chemiluminescence” in Nonisotopic Probing, Blotting and Sequencing (Kricka L. J.(ed), Academic Press, San Diego, Calif.); Nelson et al. (1994) “Application of the Hybridization Protection Assay (HPA) to PCR” in The Polymerase Chain Reaction (Mullis et al. (eds.), Birkhauser, Boston, Mass.); Weeks et al., Clin. Chem. (1983) 29:1474-1479; and Berry et al., Clin. Chem. (1988) 34:2087-2090. Current techniques allow an AE label to be placed at any location within a probe. An AE label can be directly attached to a probe using non-nucleotide-based linker arm chemistry that allows placement of the AE label at any location within the probe. Use of non-nucleotide-based linker arm chemistry is disclosed in U.S. Pat. Nos. 5,585,481 and 5,185,439.

In some instances, it is desirable to measure gene expression levels using a polynucleotide array, such as, for example, GeneChip® probe arrays (Affymetrix Inc., Santa Clara, Calif.), CodeLink® Bioarray (Amersham Biosciences Corp., Piscataway, N.J.), and the like. Probes for interrogating a tissue or cell sample are typically of sufficient length to specifically hybridize to appropriate, complementary genes or transcripts. Typically, the probes used will be at least 10, 12, 14, 16, 18, 20, or 25 nucleotides in length. In some instances, longer probes of at least 30, 40, or 50 nucleotides can be used. Genes examined using an array can include all of the genes present in an organism or a subset of sufficient size to distinguish modulation of gene expression levels in accordance with the degree of resolution and/or confidence desired. The methodology described herein can be used for determining the size of a subset of genes desirable for this purpose.

Target amplification methods (e.g., polymerase chain reaction (“PCR”) amplification of cDNA using Taqman® polymerase or other enzymatic methods), signal amplification methods (e.g., employing highly-labeled probes, chromogenic enzymes, and the like), or both, can be used to determine expression levels of various genes. Gene expression levels can also be determined using transcription-mediated amplification (“TMA”), as, for example, disclosed in U.S. Pat. No. 5,399,491, the disclosure of which is incorporated herein by reference in its entirety. As an example of a typical assay, an isolated nucleic acid sample is mixed with a buffer concentrate containing a buffer, salts, magnesium, nucleotide triphosphates, primers, dithiothreitol, and spermidine. The reaction is optionally incubated at about 100° C. for approximately two minutes to denature any secondary structure. After cooling to room temperature, reverse transcriptase, RNA polymerase, and RNase H are added, and the mixture is incubated for two to four hours at 37° C. The reaction can then be assayed by denaturing the product, adding a probe solution, incubating for 20 minutes at 60° C., adding a solution to selectively hydrolyze unhybridized probe, incubating the reaction for six minutes at 60° C., and measuring the remaining chemiluminescence in a luminometer.

TMA provides a method of identifying target nucleic acid sequences that are present in very small amounts. Such sequences may be difficult to detect using direct assay methods. In particular, TMA is an isothermal, autocatalytic nucleic acid target amplification assay system that can provide more than a billion RNA copies of a target sequence. TMA can be performed qualitatively to accurately detect the presence or absence of a target sequence in a biological sample. TMA can also provide a quantitative measure of the amount of a target sequence over a concentration range of several orders of magnitude. TMA provides a method for autocatalytically synthesizing multiple copies of a target nucleic acid sequence without repetitive manipulation of reaction conditions such as temperature, ionic strength, and pH.

Typically, TMA includes the following operations: (a) isolate nucleic acid, including RNA, from a biological sample of interest; and (b) combine into a reaction mixture (i) the isolated nucleic acid, (ii) first and second oligonucleotide primers, where the first primer has a complexing sequence sufficiently complementary to a 3′ terminal portion of an RNA target sequence (e.g., the (+) strand) to complex therewith, the second primer has a complexing sequence sufficiently complementary to a 3′ terminal portion of a target sequence of its complement (e.g., the (−) strand) to complex therewith, and where the first primer further includes a sequence 5′ to the complexing sequence that includes a promoter, (iii) a reverse transcriptase or RNA and DNA dependent DNA polymerases, (iv) an enzyme activity that selectively degrades the RNA strand of an RNA-DNA complex (e.g., an RNase H), and (v) an RNA polymerase that recognizes the promoter.

The components of the reaction mixture may be combined stepwise or at once. The reaction mixture is incubated under conditions such that an oligonucleotide/target sequence hybrid is formed, including DNA priming and nucleic acid synthesizing conditions (e.g., including ribonucleotide triphosphates and deoxyribonucleotide triphosphates) for a period of time sufficient to provide multiple copies of the target sequence. The reaction advantageously takes place under conditions suitable for maintaining the stability of reaction components (e.g., the component enzymes) and without requiring modification or manipulation of reaction conditions during the course of the amplification reaction.

Accordingly, the reaction may take place under conditions that are substantially isothermal and include substantially constant ionic strength and pH. The reaction conveniently does not require a denaturation step to separate the RNA-DNA complex produced by the first DNA extension reaction.

Suitable DNA polymerases include reverse transcriptases, such as, for example, avian myeloblastosis virus (“AMV”) reverse transcriptase (e.g., available from Seikagaku America, Inc.) and Moloney murine leukemia virus (“MMLV”) reverse transcriptase (e.g., available from Bethesda Research Laboratories).

Promoters or promoter sequences suitable for incorporation in primers include, for example, nucleic acid sequences (e.g., naturally occurring, produced synthetically, or produced using a restriction digest) that are specifically recognized by an RNA polymerase, which recognizes and binds to the sequences and initiates the process of transcription to produce RNA transcripts. The sequences may optionally include nucleotide bases extending beyond the actual recognition site for the RNA polymerase to impart added stability or susceptibility to degradation processes or increased transcription efficiency. Examples of useful promoters include those recognized by certain bacteriophage polymerases, such as those from bacteriophage T3, T7, or SP6, or those from E. coli. These RNA polymerases are available from various commercial sources, such as, for example, New England Biolabs and Epicentre.

Some of the reverse transcriptases suitable for use in the methods described herein have an RNase H activity, such as, for example, AMV reverse transcriptase. It may, however, be desirable to add exogenous RNase H, such as, for example, E. coli RNase H, even when AMV reverse transcriptase is used. RNase H is available from Bethesda Research Laboratories.

RNA transcripts that are produced may serve as templates to produce additional copies of a target sequence through the above-described mechanisms. In some instances, amplification occurs autocatalytically without the need for repeatedly modifying or changing reaction conditions such as temperature, pH, ionic strength, and the like. As mentioned above, primers and probes may be used in PCR-based techniques to determine gene expression levels of various genes. PCR refers to a technique for amplifying a desired target nucleic acid sequence contained in a nucleic acid molecule or mixture of molecules. In PCR, a pair of primers is employed in excess to hybridize to complementary strands of a target nucleic acid sequence. The primers are each extended by a polymerase using the target nucleic acid sequence as a template. The extension products become target sequences themselves after dissociation from the original target strand. New primers are then hybridized and extended by a polymerase, and the cycle is repeated to geometrically increase the number of target sequences. A PCR method for amplifying target nucleic acid sequences in a biological sample can be performed as, for example, disclosed in irmis et al. (eds.) PCR Protocols (Academic Press, NY 1990); Taylor (1991) Polymerase chain reaction: basic principles and automation, in PCR: A Practical Approach (McPherson et al. (eds.), IRL Press, Oxford); Saiki et al. (1986) Nature 324:163; as well as in U.S. Pat. Nos. 4,683,195, 4,683,202 and 4,889,818, the disclosures of which are incorporated herein by reference in their entireties.

PCR typically uses relatively short oligonucleotide primers that flank a target sequence to be amplified. The primers can be oriented such that their 3′ ends face each other with each primer extending toward the other. A polynucleotide sample is extracted and denatured, preferably by heat, and hybridized with first and second primers that are present in molar excess. Polymerization is catalyzed in the presence of the four deoxyribonucleotide triphosphates (e.g., dNTPs—dATP, dGTP, dCTP, and dTTP) using a primer- and template-dependent polynucleotide polymerizing agent, such as any enzyme capable of producing primer extension products. Examples of polynucleotide polymerizing agents include E. coli DNA polymerase I, Klenow fragment of DNA polymerase I, T4 DNA polymerase, thermostable DNA polymerases isolated from Thermus aquaticus (Taq) (e.g., available from Perkin Elmer), Thermus thermophilus (e.g., available from United States Biochemicals), Bacillus stereothermophilus (e.g., available from Bio-Rad), and Thermococcus litoralis (e.g., “Vent” polymerase available from New England Biolabs). Polymerization results in two “long products” that contain respective primers at their 5′ ends covalently linked to the newly synthesized complements of the original strands. The reaction mixture is then returned to polymerizing conditions by, for example, lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, two additional long products are produced, and a number of short products are produced equal to the number of long and short products remaining at the end of the previous cycle. Thus, the number of short products containing the target sequence grows exponentially with each cycle. Desirably, PCR is carried out with a commercially available thermal cycler, such as one available from Perkin Elmer.

RNAs may be amplified by reverse transcribing mRNA into cDNA and then performing PCR (“RT-PCR”) as described above. Alternatively, a single enzyme may be used for both steps as, for example, disclosed in U.S. Pat. No. 5,322,770. mRNA may also be reverse transcribed into CDNA, followed by asymmetric gap ligase chain reaction (“RT-AGLCR”) as, for example, disclosed in Marshall et al. (1994) PCR Meth. App. 4:80-84.

In some instances, a fluorogenic 5′ nuclease assay, known as the TaqMan™ assay (Perkin-Elmer), can be a powerful and versatile PCR-based detection system for target nucleic acid sequences. Primers and probes can be used in TaqMan™ analyses. Analysis can be performned in conjunction with thermal cycling by monitoring the generation of fluorescence signals. Such assay system dispenses with the need for gel electrophoretic analysis and has the capability to generate quantitative data to determine the number of target copies.

A fluorogenic 5′ nuclease assay can be conveniently performed using, for example, AmpliTaq Gold™ DNA polymerase, which has endogenous 5′ nuclease activity and is used to digest an internal oligonucleotide probe labeled with both a fluorescent reporter dye and a quencher (e.g., as disclosed in Holland et al., Proc. Natl. Acad. Sci. USA (1991) 88:7276-7280 and Lee et al., Nuci. Acids Res. (1993) 21:3761-3766). Assay results are detected by measuring changes in fluorescence that occur during an amplification cycle as the fluorescent probe is digested, which uncouples the dye and quencher labels and causes an increase in fluorescent signal that is proportional to the amplification of target DNA. Additional discussion of the TaqMan™ assay, reagents, and conditions can be found in Holland et al., Proc. Natl. Acad. Sci, U.S.A. (1991) 88:7276-7280; U.S. Pat. Nos. 5,538,848, 5,723,591, and 5,876,930, the disclosures of which are incorporated herein by reference in their entireties.

Amplification products can be detected in solution or using solid supports. A TaqMan™ probe can be designed to hybridize to a target sequence within a desired PCR product. The 5′ end of the TaqMan™ probe typically contains a fluorescent reporter dye. The 3′ end of the probe is typically blocked to prevent probe extension and contains a dye that will quench the fluorescence of the 5′ fluorescent label. During subsequent amplification, the 5′ fluorescent label is cleaved off if a polymerase with 5′ exonuclease activity is present in the reaction. Excision of the 5′ fluorescent label results in an increase in fluorescence that can be detected. In particular, the probe has at least one single-stranded conformation when unhybridized, such that the quencher molecule is near enough to the reporter molecule to quench the fluorescence of the reporter molecule. The probe also has at least one conformation when hybridized to a target sequence, such that the quencher molecule is not positioned close enough to the reporter molecule to quench the fluorescence of the reporter molecule. By adopting these hybridized and unhybridized conformations, the reporter molecule and quencher molecule on the probe exhibit different fluorescence signal intensities when the probe is hybridized and unhybridized. As a result, it is possible to determine whether the probe is hybridized or unhybridized based on a change in the fluorescence intensity of the reporter molecule, the quencher molecule, or a combination thereof. In addition, the probe can be designed such that the quencher molecule quenches the reporter molecule when the probe is not hybridized. As a result, the probe can be designed such that the reporter molecule exhibits limited fluorescence unless the probe is either hybridized or digested.

Ligase Chain Reaction (“LCR”) is another method for nucleic acid amplification and detection of gene expression levels. In LCR, probe pairs are used that include two primary (e.g., first and second) and two secondary (e.g., third and fourth) probes, all of which are typically used in molar excess relative to a target sequence. The first probe hybridizes to a first segment of the target sequence, and the second probe hybridizes to a second segment of the target sequence. The first and second segments are typically contiguous, such that the primary probes abut one another in a 5′ phosphate-3′ hydroxyl relationship. Thus, a ligase can covalently fuse or ligate the two probes into a fused product. In addition, a third probe can hybridize to a portion of the first probe, and a fourth probe can hybridize to a portion of the second probe in a similar abutting fashion. If the target sequence is initially double-stranded, the secondary probes can also hybridize to the target complement in the first instance. Once the ligated strand of primary probes is separated from the target sequence, it will hybridize with the third and fourth probes, which can be ligated to form a complementary and secondary ligated product. By repeated cycles of hybridization and ligation, amplification of the target sequence is achieved. Additional discussion of LCR can be found in European Publication No. 320,308, published Jun. 16, 1989; and European Publication No. 439,182, published Jul. 31, 1991.

A method of detecting the level of expression of a gene involves the use of target sequence-specific oligonucleotide probes. The probes may be used in hybridization protection assays (“HPA”). In particular, the probes can be conveniently labeled with AE, which is a highly chemiluminescent molecule. An AE molecule can be attached to a probe using a non-nucleotide-based linker arm chemistry that allows placement of the label at any location within the probe. Chemiluminescence is triggered by reaction with alkaline hydrogen peroxide, which reaction yields an excited N-methyl acridone that subsequently collapses to ground state with the emission of a photon. Additionally, AE causes ester hydrolysis, which yields a nonchemiluminescent-methyl acridinium carboxylic acid.

When an AE molecule is covalently attached to a nucleic acid probe, hydrolysis typically occurs rapidly under mildly alkaline conditions. When the AE-labeled probe is complementary (e.g., exactly complementary) to a target nucleic acid, the rate of AE hydrolysis is typically greatly reduced. Thus, hybridized and unhybridized AE-labeled probe can be detected directly in solution, without the need for physical separation. MPA typically includes the following operations. Initially, an AE-labeled probe is hybridized with a target nucleic acid sequence in solution for about 15 to about 30 minutes. A mild alkaline solution is then added, and AE coupled to the unhybridized probe is hydrolyzed. This reaction takes approximately 5 to 10 minutes. The remaining hybrid-associated AE is detected as a measure of the amount of target sequence present. This operation takes approximately 2 to 5 seconds. Desirably, the differential hydrolysis operation is conducted at the same temperature as the hybridization operation, typically at 50 to 70° C. Alternatively, a second differential hydrolysis operation may be conducted at room temperature. The second differential hydrolysis operation allows elevated pHs to be used (e.g., pH in the range of 10-11), which yields larger differences in the rate of hydrolysis between hybridized and unhybridized AE-labeled probe. Additional discussion of HPA can be found in U.S. Pat. Nos. 6,004,745, 5,948,899, and 5,283,174, the disclosures of which are incorporated herein by reference in their entireties.

Nucleic acid sequence-based amplification (“NASBA”) may also be used for determining the expression level of various genes. NASBA is a promoter-directed, enzymatic process that induces in vitro continuous, homogeneous, and isothermal amplification of a specific nucleic acid to provide RNA copies of the nucleic acid. Reagents for conducting NASBA include a first DNA primer with a 5′ tail including a promoter, a second DNA primer, reverse transcriptase, RNase-H, T7 RNA polymerase, NTP's, and dNTP's. Using NASBA, large amounts of single-stranded RNA can be generated from either single-stranded RNA or DNA or from double-stranded DNA. When RNA is to be amplified, single-stranded RNA (“ssRNA”) serves as a template for the synthesis of a first DNA strand via elongation of a first primer containing an RNA polymerase recognition site. This first DNA strand in turn serves as the template for the synthesis of a second complementary DNA strand via elongation of a second primer, resulting in a double-stranded active RNA-polymerase promoter. The second DNA strand serves as a template for the synthesis of large amounts of the first template (i.e., the ssRNA) with the aid of a RNA polymerase. Additional discussion of NASBA can be found in Guatelli et al. (1990) Proc. Natl. Acad. Sci. USA 87:1874-1878; Compton, J. Nature 350:91-92; European Patent 329,822; International Publication No. WO 91/02814; and U.S. Pat. Nos. 6,063,603, 5,554,517 and 5,409,818; the disclosures of which are incorporated herein by reference in their entireties.

Other amplification and detection methods that can be utilized include, for example, Q-beta amplification, strand displacement amplification (e.g., as disclosed in Walker et al. Clin. Chem. 42:9-13 and European Patent Application No. 684,315), and target mediated amplification (e.g., as disclosed in International Publication No. WO 93/22461).

Many of the methods described above rely on complementarity between a probe or primer and a target sequence. When single-stranded DNA (“ssDNA”) molecules form hybrids, the base sequence complementarity of the two strands need not be perfect. Poorly matched hybrids (i.e., hybrids in which only some of the nucleotides in each strand are aligned with their complementary bases so as to form hydrogen bonds) can form at low temperatures. As temperature is raised or salt concentration is lowered, complementary base-paired regions within poorer hybrids tend to dissociate due to the fact that there is not enough total hydrogen bond formation within the entire duplex molecule to hold the two strands together. The temperature and/or salt concentrations may be changed-progressively to create conditions where an increasing percentage of complementary base pair matches is required in order for hybrid duplexes to remain intact. In some instances, a set of conditions may be reached at which only perfectly matched hybrids can exist as duplexes. Above this stringency level, even perfectly matched duplexes will tend to dissociate. The stringency conditions for each unique fragment of double-stranded DNA (“dsDNA”) in a mixture of DNA depend on its unique base pair composition. The degree to which hybridization conditions require perfect base pair complementarity for hybrid duplexes to persist is referred to as a “stringency of hybridization.” Low stringency conditions are those that permit the formation of duplexes having some degree of mismatched bases. High stringency conditions are those that require near-perfect base pair complementarity for duplexes to persist. Manipulation of stringency conditions can be important for the optimization of sequence specific assays. It will be appreciated that perfect base pair complementarity is not required in many situations.

In the amplification-based methods described above, once primers or probes have been sufficiently extended and/or ligated, they can be separated from a target sequence by, for example, heating the reaction mixture to a “melt temperature” to dissociate complementary nucleic acid strands. Thus, a sequence complementary to the target sequence is formed. A new amplification cycle can then take place to further amplify the number of target sequences. In particular, the new amplification cycle can include separating any double-stranded sequences, allowing primers or probes to hybridize to their respective target sequences, extending and/or ligating the hybridized primers or probes, and re-separating. Complementary sequences that are generated by amplification cycles can serve as templates for primer extension or for filling a gap of two probes to further amplify the number of target sequences. Typically, a reaction mixture is cycled between 20 and 100 times, more typically between 25 and 50 times. In this manner, multiple copies of the target sequence and its complementary sequence can be produced. Thus, primers can initiate amplification of the target sequence when it is present under amplification conditions.

A “melting temperature” or “Tₘ” of dsDNA refers to the temperature at which half of the helical structure of the dsDNA is lost due to heating or other dissociation of hydrogen bonding between base pairs (e.g., by acid treatment, alkali treatment, or the like). The Tₘ of a DNA molecule typically depends on its length and on its base composition. DNA molecules rich in GC base pairs tend to have a higher Tₘ than those having an abundance of AT base pairs. Separated complementary strands of DNA tend to spontaneously associate or anneal to form duplex DNA when the temperature is lowered below the Tₘ. The highest rate of nucleic acid hybridization typically occurs approximately 25° C. below the Tₘ. In some instances, the Tₘ may be estimated using the following relationship: Tₘ=69.3+0.41 (GC)% (e.g., as disclosed in Marmur et al. (1962) J. Mol. Biol 5:109-118).

In some instances, two or more assays can be performed. For example, if a first assay used TMA to amplify nucleic acids for detection, then an alternative nucleic acid testing (“NAT”) assay can be performed by, for example, using PCR amplification, RT-PCR, or the like. It should be recognized that the design of assays described herein may be subject to a certain degree of variation, and many variations can be used. The above descriptions are merely provided as guidance, and one of skill in the art can readily modify the described protocols using techniques known in the art.

Detection of target sequences, both amplified and non-amplified, may be performed using a variety of heterogeneous and homogeneous detection formats. Examples of heterogeneous detection formats include those disclosed in U.S. Pat. No. 5,273,882 to Snitman et al., U.S. Pat. No. 5,124,246 to Urdea et al., U.S. Pat. No. 5,185,243 to Ullman et al., and U.S. Pat. No. 4,581,333 to Kourilsky et al., the disclosures of which are incorporated herein by reference in their entireties. Examples of homogeneous detection formats include those disclosed in U.S. Patent No. 5,582,989 to Caskey et al. and U.S. Pat. No. 5,210,015 to Gelfand et al., the disclosures of which are incorporated herein by reference in their entireties. It is also contemplated that detection methods can include use of multiple probes in hybridization assays to improve sensitivity and amplification of a target signal. Examples of use of multiple probes include those disclosed in U.S. Pat. No. 5,582,989 to Caskey et al. and U.S. Pat. No. 5,210,015 to Gelfand et al.

High Throughput Techniques for Screening Compounds

Protocols have been developed to rapidly evaluate multiple compounds in a particular bioassay system as well as a compound in multiple bioassay systems. Such protocols for evaluating compounds can be referred to as high throughput screening (“HTS”). In one typical protocol, HTS involves the dispersal of a compound into a well of a multiwell cluster plate, such as, for example, a 96-well plate or higher format plate in the form of a 384-, 864-, or 1536-well plate. The effect of the compound is evaluated on a bioassay system in which the compound is being tested. The “throughput” of HTS (i.e., the combination of the number of compounds that can be screened and the number of bioassay systems against which compounds can be screened) may be determined based on a number of factors, including, for example: (1) one assay can be performed per well; (2) if conventional dye molecules are used to monitor the effect of a compound, multiple excitation sources can be required if multiple dye molecules are used; and (3) as the well size becomes small (e.g., a 1536-well plate can accept about 5 μl of total assay volume), consistent dispensing of individual components into a well can be difficult, and the amount of signal generated by each assay can be decreased and can scale with the volume of the assay.

In some instances, a 1536-well plate is a physical segregation of sixteen assays within a single 96-well plate format. It may be desirable to multiplex 16 assays into a single well of a 96-well plate. This would result in greater ease of dispensing reagents into the wells and in high signal output per well. In addition, performing multiple assays in a single well can allow simultaneous determination of the potential of a compound to affect a plurality of target bioassay systems. Using HTS strategies, a single compound can be screened for various biological activities (e.g., as a protease inhibitor, an inflammation inhibitor, an anti-asthmatic, and the like) in a single assay.

For certain applications, an HTS assay using emission labels as multiplexed detection reagents is provided. The HTS assay can be performed in the presence of various concentrations of a compound. Emission can be monitored as an indication of the effect of the compound on the assay system. For example, fluorescence reading using a labeled ligand or receptor to monitor its binding to a bead-bound receptor or ligand may be used to measure emission associated with various beads. Emission associated with the beads can be a function of the concentration of the compound and, thus, can serve to measure the effect of the compound on the bioassay system. In addition, a multicolor scintillant can be used to detect the binding of a radiolabeled ligand or receptor with a labeled receptor or ligand. A decrease in scintillation can be one result of inhibition by the compound of the ligand-receptor pair binding.

Matrix Representation of Gene Expression Datasets

Gene expression data, whether obtained from array experiments or otherwise, can be represented in the form of a set of gene expression matrices or tables. In some instances, gene expression datasets obtained from a set of biological samples can be used to form a set of two-dimensional gene expression matrices. Each row of a gene expression matrix can be associated with a particular gene, and each column of the gene expression matrix can be associated with a particular set of measurements. Alternatively, each row of a gene expression matrix can be associated with a particular set of measurements, and each column of the gene expression matrix can be associated with a particular gene.

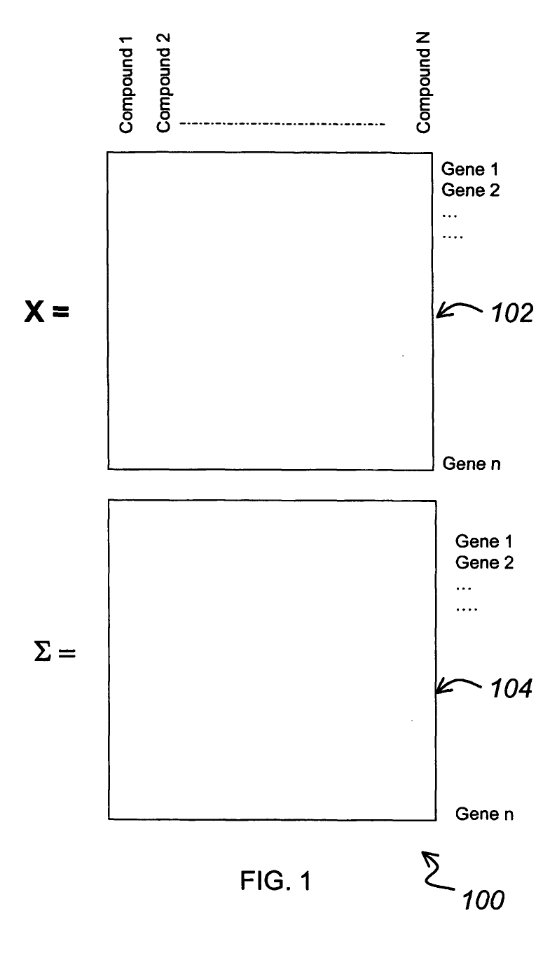

FIG. 1 illustrates an example of a set of two gene expression matrices 100, according to an embodiment of the invention. As shown in FIG. 1, the set of gene expression matrices 100 includes a matrix X 102 and a matrix Σ 104.

The matrix X 102 corresponds to a n×N matrix of gene expression levels and can be referred to as a “gene expression level matrix.” In the illustrated embodiment, each row of the matrix X 102 is associated with a particular gene of a set of genes (i.e., gene 1 through gene n), and each column of the matrix X 102 is associated with a set of measurements for a particular compound of a set of compounds (i.e., compound 1 through compound N). The matrix X 102 includes various data values organized with respect to the n rows and N columns. In the illustrated embodiment, each data value included in the matrix X 102 indicates a typical expression level of a particular gene in response to exposure to a particular compound.

The matrix Σ 104 corresponds to a n×N matrix of gene expression intervals and can be referred to as a “gene expression interval matrix.” As discussed for the matrix X 102, each row of the matrix Σ 104 is associated with a particular gene of a set of genes (i.e., gene 1 through gene n), and each column of the matrix Σ 104 is associated with a set of measurements for a particular compound of a set of compounds (i.e., compound 1 through compound N). The matrix Σ 104 includes various data values organized with respect to the n rows and N columns. In the illustrated embodiment, each data value included in the matrix Σ 104 indicates a range of variation of an expression level of a particular gene in response to exposure to a particular compound.

In the illustrated embodiment, the set of compounds can include different classes of compounds, and the matrices X 102 and Σ 104 can include various sub-matrices associated with the different classes of compounds. For example, a first class of compounds (e.g., class+) can include N₊ compounds (e.g., compound 1 through compound N₊, where N₊<N), and a second class of compounds (e.g., class-) can include N₋ compounds (e.g., compound N₊+1 through compound N, where N=N₊+N₋). Class+ can include compounds that share a particular biological activity, while class− can include compounds that do not share that biological activity or that can share a different biological activity. For example, class+ can include various compounds that share a primary biological activity, while class− can include various compounds that do not share that primary biological activity. The number of compounds included in class+ may be based on the number of related compounds available and is typically at least 2 (e.g., between 2 and 200, such as between 2 and 100, between 2 and 50, or between 10 and 200). Similarly, the number of compounds included in class− is typically at least 2 (e.g., between 2 and 200, such as between 2 and 100, between 2 and 50, or between 10 and 200). In some instances, class+ may include a smaller number of compounds than class−. For example, class+ can include various compounds that share a particular biological activity, while class− can include various remaining compounds of the set of compounds (e.g., all remaining compounds of the set of compounds).

When analyzing gene expression data obtained from measurements for a number of genes (e.g., several hundred or more genes), it is sometimes desirable to select genes that exhibit greater changes in gene expression levels. With reference to FIG. 1, selection of genes that exhibit greater changes in gene expression levels allows the number of dimensions n to be reduced and improves computational efficiency and ease of interpretation of results. For typical stimuli, a small number of genes may respond to a high degree (e.g., an increase or decrease in gene expression level by a factor of five or more), and between approximately 100 to 500 genes may exhibit a lesser but still detectable response. Many genes typically do not significantly respond and can often be excluded from further analysis without substantial loss of information. Methods for reducing large datasets based on gene impact is described in U.S. patent application Ser. No. 60/565,793 filed Apr. 26, 2004 (entitled “Reduced Subsets of Multivariate Data Useful for Diagnostic Development”) which is hereby incorporated by reference herein for all purposes.

Derivation of Classitication Rules (i.e. Signatures) from Datasets

In the general method of the present invention, classification rules may be mined from a large multidimensional (i.e. multivariate) dataset comprising gene expression data by first labeling the full dataset according to known classifications and then applying an algorithm to the full dataset that produces a linear classifier for each particular classification question. Each signature so generated is then cross-validated using a standard split sample procedure.

The initial questions used to classify (i.e. the classification questions) a large multivariate dataset may be of any type susceptible to yielding a yes or no answer. The general form of such questions is: “Is the unknown a member of the class or does it belong with everything else outside the class?” For example, in the area of chemogenomic datasets, classification questions may include “mode-of-action” questions such as “All treatments with drugs belonging to a particular structural class versus the rest of the treatments” or pathology questions such as “All treatments resulting in a measurable pathology versus all other treatments.” In the specific case of a chemogenomic dataset based on gene expression, it is preferred that the classification questions are further categorized based on the tissue source of the gene expression data. Similarly, it may be helpful to subdivide the dataset so that specific classification questions are limited to particular subsets of data (e.g. data obtained at a certain time or dose of test compound). Typically, the significance of subdividing data within large datasets becomes apparent upon initial attempts to classify the complete dataset.

Labels are assigned to each individual (e.g. each compound treatment) in the dataset according to a rigorous rule-based system. The +1 label indicates that a treatment falls in the class of interest, while a −1 label indicates that the variable is outside the class. Information used in assigning labels to the various individuals to classify may include annotations from the literature related to the dataset (e.g. known information regarding the compounds used in the treatment), or experimental measurements on the exact same animals (e.g., results of clinical chemistry or histopathology assays performed on the same animal).

More specifically, in the method of the present invention, a classification rule for gene expression data may be derived in accordance with a setup as follows: n represents the number of genes for which measurements are made, N represents the number of compounds, X represents a n×N gene expression level matrix (e.g., the matrix X 102), Σ represents a n×N gene expression interval matrix (e.g., the matrix Σ104), ρ≧0, and y ∈R^{N}. The parameter ρ represents a parameter (e.g., a global parameter) that sets ranges of variation of gene expression levels, and, in some instances, the parameter ρ can be set as 1. The N compounds include N₊ compounds included in class+ and N₋ compounds included in class−. In the present setup, the N₊ compounds include compounds that share a known or predicted biological activity associated with class+, and the N₋ compounds include compounds that do not share that biological activity. The vector y represents a labeling vector with Ncomponents. The components of the labeling vectory y serve as indicators of classes of compounds to which the N compounds belong. Depending on the particular application, the components of y can take on values of 0 and 1 (or ±1) for class+ and class−, respectively. Thus, for example, an i^{th} component of y can take on a value of 0 or 1 depending on whether an i^{th} compound belongs to class+ or class−.

In accordance with this setup, X, Σ, and ρ form an interval matrix model for a n×N matrix Z via a hyper-rectangle in the space of n×N matrices:
χ(ρ)={Z∈R^{n×N}:X−ρΣ∈≦Z≦X+ρΣ}, (1)
where inequalities are understood to be component wise. Using the interval matrix model, a linear classification rule can be derived. In particular, the gene expression data included in the interval matrix model can be used as a training set to derive the linear classification rule. Once derived, the linear classification rule can be used to assign a compound having an unknown biological activity to class+ or class−. Based on such assignment, a biological activity of the compound can be predicted.

A linear classification rule is typically associated with a linear classification function given by: w^{T}x+b, where w∈Rⁿ, x∈Rⁿ, b is a scalar, and w^{T}x represents a scalar dot product between w and x (i.e., w₁ x₁ +w₂ x₂ + . . . +wₙ xₙ). Here, x represents a multi-dimensional data vector to be classified, w represents a classifier vector, and w and b collectively include a set of classifiers of the linear classification function. The multi-dimensional data vector x can correspond to a set of gene expression levels in response to a compound and can be classified to a particular category based on the sign, magnitude, or both, of the linear classification function. For example, once the classifiers are derived in accordance with the methodology described herein, the compound can be assigned to class+ or class− based on the sign of the linear classification function. As a result of its linearity, a linear classification function can facilitate interpretation of results by, for example, allowing identification of a subset of genes that may play a greater role in a biological activity or a biological state. In particular, the n components of the classifier vector w can represent weights associated with respective genes of the n genes, and the subset of genes can be identified based on relative magnitudes of the n components.

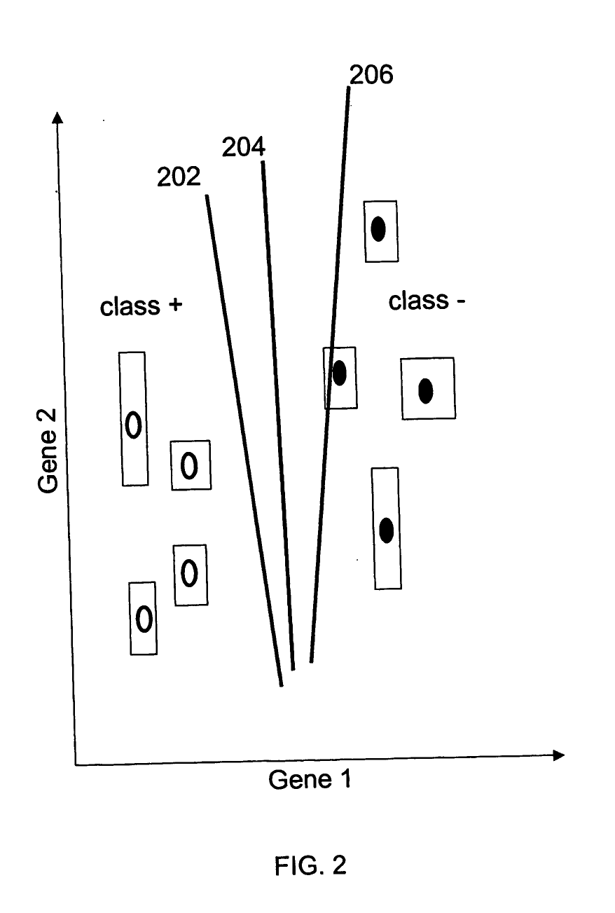

A linear classification function can be further understood with reference to FIG. 2, which illustrates gene expression data plotted in a multi-dimensional space, according to an embodiment of the invention. The gene expression data can be used as a training set to derive a linear classification function. While two dimensions associated with genes 1 and 2 are shown in FIG. 2, it should be recognized that the multi-dimensional space, in general, can include n dimensions. As shown in FIG. 2, gene expression datasets associated with compounds in class+ and class− are plotted in the multi-dimensional space. In the illustrated embodiment, compounds belonging to each class of compounds can share a biological activity and can produce similar gene expression responses. Thus, as shown in FIG. 2, gene expression datasets associated with compounds in class+ and class− tend to cluster at respective regions in the multi-dimensional space. Typical gene expression levels in response to various compounds in class+ (e.g., as specified by the matrix X) are represented by the open circles, while typical gene expression levels in response to various compounds in class− (e.g., as specified by the matrix X) are represented by the solid circles. Referring to FIG. 2, ranges of variation of gene expression levels in response to the various compounds (e.g., as specified by the matrix Σ and ρ) are represented by the rectangular boxes surrounding the open and solid circles. In the illustrated embodiment, the sides of a box can have the same length or different lengths, depending on whether genes expression intervals along the two dimensions are the same or different.

With reference to FIG. 2, various linear classification functions 202, 204, and 206 having different classifiers are plotted in the multi-dimensional space. In the illustrated embodiment, the linear classification functions 202, 204, and 206 are represented as lines in the multi-dimensional space. However, it should be recognized that the linear classification functions 202, 204, and 206, in general, can be represented as hyperplanes. As shown in FIG. 2, the linear classification functions 202, 204, and 206 separate the two clusters of gene expression datasets with varying degrees of performance. In particular, the linear classification finctions 202 and 204 adequately separate the two clusters of gene expression datasets even if gene expression levels take on different values within respective ranges of variation. On the other hand, the linear classification function 206 may produce misclassification errors in certain situations. In particular, the linear classification function 206 may improperly classify a set of gene expression levels as being associated with class+ if gene expression levels take on certain values within respective ranges of variation.

Loss Functions and the “Worse Case” Methodology

In one preferred embodiment, the present invention provides a methodology wherein linear classifiers for large datasets may be derived by minimizing a loss function. A loss function L can be defined as a measure of the performance of a linear classification function on a training set. The loss function L can depend on w and b, the matrix X(or the matrix Z in accordance with the interval matrix model), and the labeling vector y. Typically, a smaller value of the loss function is associated with a better performance on the training set. Thus, the present invention describes a “worse case” methodology wherein the loss function is reduced by minimization procedures. As described below, the present invention describes in detail the “worse case” methodology as employed with three distinct types of loss function: (1) support vector machine (SVM); (2) logistic regression (LR); and (3) minimax probability machines (MPM).

1. SVM-based Methodologies and Algorithms

In this section, application of a “worse-case” methodology to the loss function L_{SVM} is discussed. The “worse-case” methodology can be represented as:

$${L}_{SVM} ( w , b , X , y ) = {\sum }_{i = 1}^{N} {( 1 - {y}_{i} ( {w}^{T} {x}_{i} + b ) )}_{+} , \qquad ( A \text{-} 1 )$$

where ( . . . )₊ denotes a positive part of a quantity included in the parenthesis, and xᵢ represents an i^{th} column of the matrix X. In the above relation, the labeling vectory is a ±1 labeling vector. The loss function L_{SVM} represents the number of misclassification errors on the training set and is convex in w and b.

For the loss function L_{SVM}, a “worst-case” loss function can be represented as:

$$\begin{matrix}{L}_{SVM}^{wc} ( w , b ) = {max}_{Z ∈ X} {\sum }_{i = 1}^{N} {( 1 - {y}_{i} ( {w}^{T} {z}_{i} + b ) )}_{+} \\ = {\sum }_{i = 1}^{N} {( 1 - {y}_{i} ( {w}^{T} {x}_{i} + b ) + {ρσ}_{i}^{T} \vert w \vert )}_{+} .\end{matrix} \qquad ( A \text{-} 2 )$$

Hence, the “worse-case” methodology can be represented as a linear program:

$${min}_{w , b} {\sum }_{i = 1}^{N} {e}_{i} \text{:} {y}_{i} ( {w}^{T} {\hat{x}}_{i} + b ) \geq 1 - {e}_{i} + {ρσ}_{i}^{T} \vert w \vert , \\ {e}_{i} \geq 0 , \\ i = 1 , … , N . \qquad ( A \text{-} 3 )$$

For the slack variable e set to zero, the constraints in the above relation specify a hyperplane defined by w and b that perfectly separates gene expression data in respective classes, irrespective of their values in hyper-rectangles of shapes and sizes determined by the matrix Σ and ρ.

An upper bound for the “worst-case” loss function can be defined by exploiting its convexity as follows:

$$\begin{matrix}{L}_{SVM}^{wc} ( w , b ) \leq {\sum }_{i = 1}^{N} {( 1 - {y}_{i} ( {w}^{T} {x}_{i} + b ) )}_{+} + {ρσ}^{T} \vert w \vert , \\ where & ( A \text{-} 4 ) \\ σ = {\sum }_{i = 1}^{N} {σ}_{i} & ( A \text{-} 5 )\end{matrix}$$

represents a vector with n components. Each component of the vector σ corresponds to a sum of gene expression intervals for a particular gene with respect to the N compounds.

The upper bound can be used as an approximation for the “worst-case” loss function, and minimizing the upper bound also leads to a linear program:

$${min}_{w , b} {\sum }_{i = 1}^{N} {e}_{i} + {ρσ}^{T} \vert w \vert \text{:} {y}_{i} ( {w}^{T} {x}_{i} + b ) \geq 1 - {e}_{i} , \\ {e}_{i} \geq 0 , \\ i = 1 , … , N . \qquad ( A \text{-} 6 )$$

Relation (A-6) can be interpreted as an l₁-normn regularization problem with regularization parameters that are expressed in terms of the interval matrix model. In particular, relation (A-6) represents a generalization of a linear program-support vector machine, namely:

$${min}_{w , b} {\sum }_{i = 1}^{n} \vert {w}_{i} \vert + C {\sum }_{i = 1}^{N} {e}_{i} \text{:} {y}_{i} ( {w}^{T} {x}_{I} + b ) \geq 1 - {e}_{i} , \\ i = 1 , … , N , \qquad ( A \text{-} 7 )$$

where C=1/ρ, and σ is assumed to be all ones. Solutions to a linear program-support vector machine can be obtained as, for example, disclosed in P. S. Bradley and O. L. Mangasarian, Massive Data Discrimination via Linear Support Vector Machines, Optimization Methods and Software, 13(1):1-10 (2000), the disclosure of which is incorporated herein by reference in its entirety.

The upper bound for the “worst-case” loss ftmction can be understood as follows. Assuming that the slack variable e is set to zero, minimizing the upper bound for the “worst-case” loss function can be represented as:

$${min}_{w , b} {σ}^{T} \vert w \vert \text{:} {y}_{i} ( {w}^{T} {x}_{i} + b ) \geq 1 , \\ i = 1 , … , N . \qquad ( A \text{-} 8 )$$

By homogeneity, σ|w| can be set to 1/ρ, and the parameter σ can be maximized to derive a hyperplane that maintains a perfect separation between gene expression data in respective classes, irrespective of their values in hyper-rectangles of shapes and sizes determined by the matrix χ(ρ) This leads to the relation:

$${max}_{w , b} ρ \text{:} {y}_{i} ( {w}^{T} {x}_{i} + b ) \geq {ρσ}^{T} \vert w \vert , \\ i = 1 , … , N . \qquad ( A \text{-} 9 )$$

Therefore, relation (A-6) can be interpreted as a problem that involves increasing the level of robustness with respect to possible realizations of gene expression data while maintaining an adequate level of separation between gene expression data in respective classes.

Additional discussion regarding the loss function L_{SVM} can be found in e.g., N. Cristianini and J. Shawe-Taylor, “An Introduction to Support Vector Machines” (Cambridge University Press, Cambridge, U.K., 2000), the disclosure of which is incorporated herein by reference in its entirety.

2. LR-based Methodologies and Algorithms

In this section, application of a “worse-case” methodology to the loss function L_{LR} is discussed. The “worse-case” methodology can be represented as:

$${L}_{LR} ( w , b , X , y ) = {\sum }_{i = 1}^{N} ( log ( 1 + {ⅇ}^{{w}^{T} {x}_{i} + b} ) - {y}_{i} ( {w}^{T} {x}_{i} + b ) ) , \qquad ( B \text{-} 1 )$$

where y is a 0, 1 labeling vector, and xᵢ again represents an i^{th} column of the matrix X. The loss function L_{LR} is based on a maximum-likelihood approach that uses a parametric probabilistic model for the distribution of the labeling vectory. As with the loss function L_{SVM}, the loss function L_{LR} is convex in w and b.

Primal Problem

Relation (B-1) represents a convex optimization problem that involves the maximization of a concave function. The relation can be reduced to a finite-dimensional problem as follows. Assuming τ∈R, φ_{y}(τ)=yτ−log(1+e^{τ}), φ₊ corresponds to φ_{y}(τ) with y set to 1, φ₋ corresponds to φ_{y}(τ) with y set to 0, it can be shown that:

$$\begin{matrix}ψ = {max}_{w , b} {\sum }_{i = 1}^{N} {min}_{{z}_{i}} \{ {ϕ}_{{y}_{i}} ( {w}^{T} {z}_{i} + b ) \text{:} {x}_{i} - {σ}_{i} \leq {z}_{i} \leq {x}_{i} + {σ}_{i} \} \\ = {max}_{w , b} {\sum }_{i = 1}^{N} {min}_{τ} \{ {ϕ}_{{y}_{i}} ( τ + b ) \text{:} {x}_{i}^{T} w - {σ}_{i}^{T} \vert w \vert \leq τ \leq {x}_{i}^{T} w + {σ}_{i}^{T} \vert w \vert \} .\end{matrix} \qquad \text{(B-2)}$$

Since φ₋ is decreasing, and φ₊ increasing, it follows that:

$${min}_{τ} \{ {ϕ}_{y} ( τ + b ) \text{:} {x}^{T} w - σ T \vert w \vert \leq τ \leq {x}^{T} w + {σ}^{T} \vert w \vert \} = \{ \begin{matrix}{ϕ}_{-} ( {x}^{T} w + {σ}^{T} \vert w \vert + b ) & if y = 0 , \\ {ϕ}_{+} ( {x}^{T} w - {σ}^{T} \vert w \vert + b ) & otherwise .\end{matrix} \qquad ( B \text{-} 3 )$$

Using relation (B-3), it can be shown that:

$$ψ = {max}_{w , b} {\sum }_{i \text{:} {y}_{i} = 0} {ϕ}_{-} ( {x}_{i}^{T} w + {σ}_{i}^{T} \vert w \vert + b ) + {\sum }_{i \text{:} {y}_{i} = 1} {ϕ}_{+} ( {x}_{i}^{T} w - {σ}_{i}^{T} \vert w \vert + b ) . \qquad ( B \text{-} 4 )$$

Using the monotonicity of φ₋ and φ₊ and introducing a new variable in relation (B-4), ψ can be represented as a finite-dimensional, convex optimization problem:

$$\begin{matrix}ψ = \begin{matrix}{max}_{w , b , t} {\sum }_{i : {y}_{i} = 0} {ϕ}_{-} ( {x}_{i}^{T} w + {σ}_{i}^{T} t + b ) + \\ {\sum }_{i : {y}_{i} = 1} {ϕ}_{+} ( {x}_{i}^{T} w - {σ}_{i}^{T} t + b ) \text{:} t \geq w , t \geq - w\end{matrix} \\ = \begin{matrix}{max}_{w , b , i} {\sum }_{i : {y}_{i} = 0} ( {y}_{i} ( {w}^{T} {x}_{i} + b - {σ}_{i}^{T} t ) - \\ log ( 1 + {ⅇ}^{{w}^{T} {x}_{i} + b + ( 1 - {2}_{{y}_{i}} ) {σ}_{i}^{T} t} ) ) \text{:} t \geq w , t \geq - w .\end{matrix}\end{matrix} \qquad ( B \text{-} 5 )$$

Relation (B-5) can be interpreted as a conventional logistic regression problem with additional sign constraints on a set of classifiers.

Dual Problem

A dual of the above problem can be interpreted in terms of maximum entropy.

Using the variables wₚ=t+w and wₙ=t−w, the following vectors and matrices can be defined:

$$ξ = ( \begin{matrix}{w}_{p} \\ {w}_{n} \\ b\end{matrix} ) , v = {\sum }_{i} {y}_{i} ( \begin{matrix}{x}_{i} - {σ}_{i} \\ - ( {x}_{i} + {σ}_{i} ) \\ 1\end{matrix} ) , {a}_{i} = ( \begin{matrix}{x}_{i} + ( 1 - {2}_{{y}_{i}} ) {σ}_{i} \\ - {x}_{i} + ( 1 - {2}_{{y}_{i}} ) {σ}_{i} \\ 1\end{matrix} ) , 1 \leq i \leq N , \\ M = ( \begin{matrix}I & 0 & 0 \\ 0 & I & 0\end{matrix} ) , A = ( \begin{matrix}{a}_{1} & ⋯ & {a}_{N}\end{matrix} ) \qquad ( B \text{-} 6 )$$

A dual problem can be represented as:

$${max}_{ξ , f} {v}^{T} ξ - {\sum }_{i = 1}^{N} log ( 1 + {ⅇ}^{{f}_{i}} ) \text{:} f = {A}^{T} ξ , M ξ \geq 0. \qquad ( B \text{-} 7 )$$

The dual problem represented in relation (B-7) is convex and strictly feasible. Also, a Lagrangean can be represented as:

$$ℒ ( ξ , f , λ , μ ) = {v}^{T} ξ - {\sum }_{i = 1}^{N} log ( 1 + {ⅇ}^{{f}_{i}} ) + {λ}^{T} ( f - {A}^{T} ξ ) + {μ}^{T} M ξ . \qquad ( B \text{-} 8 )$$

The optimization conditions yield:

$$v = A λ - {M}^{T} μ , {λ}_{i} = \frac{{ⅇ}^{{f}_{i}}}{1 + {ⅇ}^{{f}_{i}}} , i = 1 , … , N . \qquad ( B \text{-} 9 )$$

Thus, the dual problem can be represented as:

$${min}_{λ , μ} {λ}^{T} log λ + {( 1 - λ )}^{T} log ( 1 - λ ) \text{:} 0 \leq λ \leq 1 , A λ = {M}^{T} μ + v , μ \geq 0. \qquad ( B \text{-} 10 )$$

The dual problem is equivalent to the original problem as a result of the strict feasibility of the latter. Partitioning μ for the two classes as μ=(μ₊, μ₋), the condition Aλ=M^{T}μ+v can be represented as:

$${\sum }_{i} ( {x}_{i} + ( 1 - 2 {y}_{i} ) {σ}_{i} ) {λ}_{i} = {\sum }_{i} ( {x}_{i} - {σ}_{i} ) {y}_{i} + {μ}_{+} , \\ {\sum }_{i} ( - {x}_{i} + ( 1 - 2 {y}_{i} ) {σ}_{i} ) {λ}_{i} = - {\sum }_{i} ( {x}_{i} + {σ}_{i} ) {y}_{i} + {μ}_{-} , \\ {\sum }_{i} {λ}_{i} = {\sum }_{i} {y}_{i} . \qquad ( B \text{-} 11 )$$

The existence of non-negative vectors μ₊ and μ₋ such that the above conditions hold leads to the following relation:
|X(y−λ)|≦Σ|y−λ|, (B-12)
which can be recast as linear inequalities in λ when 0≦λ≦1.

Thus, the dual problem can be represented as:

$$ψ = {min}_{λ} {λ}^{T} log λ + {( 1 - λ )}^{T} log ( 1 - λ ) \text{:} \vert X ( y - λ ) \vert \leq Σ \vert y - λ \vert , \\ 0 \leq λ \leq 1 , \\ {( y - λ )}^{T} 1 = 0. \qquad ( B \text{-} 13 )$$

Relation (B-1 3) can be interpreted as a maximum entropy problem and is amenable to efficient solutions using interior-point methods, such as, for example, methods implemented in conventional convex optimization software. One example of a conventional convex optimization software is MOSEK Optimization Software available from MOSEK ApS, located in Copenhagen, Denmark.

Values for w and b can be derived by noting that ξ is a variable dual to the equality constraint Aλ=M^{T}μ+v, which means that w_{±} and b are dual to respective conditions shown in relation (B-11).

It should be recognized that, for Σ=0, the dual problem reduces to a maximum entropy problem with “exact” moment matching constraints:

$${min}_{λ} {λ}^{T} log λ + {( 1 - λ )}^{T} log ( 1 - λ ) \text{:} X ( y - λ ) = 0 , \\ 0 \leq λ \leq 1 , \\ {( y - λ )}^{T} 1 = 0 ,$$

which is the dual of a conventional logistic regression problem. Here, the constraint shown in relation (B-12) indicates that an “exact” moment match exists for at least one matrix within the uncertainty set X, namely:
∃X∈χ, X(y−λ)=0. (B-15)

This matrix can be interpreted as a “worst-case” realization of gene expression data, namely, one that yields a “worst-case” value of the likelihood function for optimized w and b. This “worst-case” matrix can be represented as:
X_{wc}=X+Σ(μ1^{T}), (B-16)
where uⱼ=(1−2y(j)sign(w(j)), and products are understood to be component wise.

Upper Bound

In some instances, an approximation may be made by maximizing a lower bound of the “worst-case” log-likelihood function. For each pair (τ, r), with r≧0, it can be shown that:
φ₋(τ+τ)≧φ₋(τ)−τ and φ₊(τ−τ)≧φ₊(τ)−τ. (B-17)

Applying these inequalities with
τ=xᵢ^{T}w, τ=σᵢ^{T}|w|≧0 (B-18)
in relation (B-4), it can be shown that:

$$\begin{matrix}\begin{matrix}ψ = {max}_{w , b} {\sum }_{i \text{:} {y}_{i} = 0} {ϕ}_{-} ( {x}_{i}^{T} w + {σ}_{i}^{T} \vert w \vert + b ) + \\ {\sum }_{i \text{:} {y}_{i} = 1} {ϕ}_{+} ( {x}_{i}^{T} w - {σ}_{i}^{T} \vert w \vert + b ) \\ \geq \underline{ψ} \text{:} \\ = {max}_{w , b} {\sum }_{i \text{:} {y}_{i} = 0} ( {ϕ}_{-} ( {x}_{i}^{T} w + b ) - {σ}_{i}^{T} \vert w \vert ) + \\ {\sum }_{i \text{:} {y}_{i} = 1} ( {ϕ}_{+} ( {x}_{i}^{T} w + b ) - {σ}_{i}^{T} \vert w \vert ) \\ = {max}_{w , b} {\sum }_{i = 1}^{N} ( {y}_{i} ( {w}^{T} {z}_{i} + b ) - log ( 1 + {ⅇ}^{{w}^{T} {z}_{i} + b} ) ) - {σ}^{T} \vert w \vert ,\end{matrix} \\ where & ( B \text{-} 19 ) \\ σ := {\sum }_{i} {σ}_{i} = Σ1 & ( B \text{-} 20 )\end{matrix}$$

represents a sum of gene expression intervals with respect to the N compounds.

Relations (B-19) and (B-20) can be interpreted as an l₁-norm regularization problem with regularization parameters that are expressed in terms of the interval matrix model. It can be shown that this problem produces sparse classifiers, typically much sparser than a conventional squared l₂-norm regularization problem.

Computing ψ can also be performed with interior-point methods for maximum entropy problems, such as, for example, methods implemented in conventional convex optimization software. In particular, a new variable can be introduced to express ψ as:

$$\underline{ψ} = {max}_{w , b} {\sum }_{i = 1}^{N} □ ( {y}_{i} ( {w}^{T} {x}_{i} + b ) - log ( 1 + {ⅇ}^{{w}^{T} {x}_{i} + b} ) ) - {σ}^{T} t : t \geq w , \\ t \geq - w . \qquad ( B \text{-} 21 )$$

Defining the vectors

$$ξ = ( \begin{matrix}w \\ b \\ t\end{matrix} ) , \\ {a}_{i} = ( \begin{matrix}{x}_{i} \\ 1 \\ 0\end{matrix} ) , \\ υ = ( \begin{matrix}{\sum }_{i} {y}_{i} {x}_{i} \\ {\sum }_{i} {y}_{i} \\ - {σ}_{i}\end{matrix} ) , \qquad ( B \text{-} 22 )$$

and the matrices A and M as shown in relation (B-6), it can be shown that relation (B-21) reads as relation (B-7), and its dual problem reads as relation (B-10). Thus, a dual problem can be represented as:

$$\underline{ψ} = {min}_{λ} {λ}^{T} log λ + {( 1 - λ )}^{T} log ( 1 - λ ) \text{:} \\ \vert X ( y - λ ) \vert \leq σ , \\ 0 \leq λ \leq 1 , \\ {( y - λ )}^{T} 1 = 0. \qquad ( B \text{-} 23 )$$

For 0≦λ<1 and assuming that Σ₃₁ and λ₋ correspond to Σ and λ with y set to 0 for class−, and Σ₊ and λ₊ correspond to Σ and λ with y set to 1 for class+, it follows that:
Σ|y−λ|=Σ₋λ₋+Σ₊(1−λ₊)≦Σ₋1+Σ₊1=Σ1=σ. (B-24)

Thus, a condition for moment matching to occur within the uncertainty set χ corresponds to |X(y−λ)|≦σ. Hence, the dual problem based on the approximation can be obtained as a relaxation (e.g., a lower bound) of the dual problem without the approximation.

Additional discussion regarding the loss function L_{LR} can be found in e.g., Trevor Hastie, Robert Tibshirani, and Jerome Friedman, “Elements of Statistical Learning: Data Mining, Inference and Prediction” (Springer-Verlag, 2001), the disclosure of which is incorporated herein by reference in its entirety.

3. MPM-based Methodologies and Algorithms

In this section, application of a “worse-case” methodology to the loss function L_{MPM} is discussed. The “worse-case” methodology can be represented as:

The minimax probability machine loss function L_{MPM} can be represented as:

$${L}_{MPM} ( w , X , y ) = \frac{\sqrt{{w}^{T} {Γ}_{+} w} + \sqrt{{w}^{T} {Γ}_{-} w}}{\vert {w}^{T} ( {\hat{x}}_{+} - {\hat{x}}_{-} ) \vert} , \qquad ( C \text{-} 1 )$$

where \hat{x}± represents empirical mean vectors for class+ and class−, respectively, and Γ± represents empirical covariance matrices for class+ and class−, respectively. The loss function L_{MPM} is convex on a hyperplane defined by w^{T}(\hat{x}₊−\hat{x}₋)=1, and the methodology described herein can be applied with respect to this hyperplane.

In general, an empirical mean vector \hat{x} and an empirical covariance matrix Γ for a set of N compounds can be represented as:

$$\hat{x} = \frac{1}{N} {\sum }_{i = 1}^{N} {x}_{i} \\ Γ = \frac{1}{N} {\sum }_{i = 1}^{N} ( {x}_{i} - \hat{x} ) {( {x}_{i} - \hat{x} )}^{T} \qquad ( C \text{-} 2 )$$

Uncertainty in gene expression data can be taken to be smoothed out when computing class means \hat{x}±.

Basic Formulation

A minimax probability machine is associated with a binary classification rule that uses class means to control misclassification error. The following assumptions can be made: x_{+ and x}_{- represent random vectors that model data from the two classes, \hat{x}}_{±, x}_{+, and x}_{- each has n components, and Γ}_{± is positive semidefinite.}

The minimax probability machine can determine a hyperplane:
H(w, b)={z|w^{T}z=b}, (C-3)

which separates the two classes with maximal probability with respect to all distributions having these mean vectors and covariance matrices. This leads to:

$${max}_{α , w , b} α \\ s . t . \\ \begin{matrix}{inf}_{{x}_{+} \sim ( {\hat{x}}_{+} , {Γ}_{+} )} Pr \{ {w}^{T} {x}_{+} \geq b \} \geq α \\ {inf}_{{x}_{-} \sim ( {\hat{x}}_{-} , {Γ}_{-} )} Pr \{ {w}^{T} {x}_{-} \leq b \} \geq α ,\end{matrix} \qquad ( C \text{-} 4 )$$

where the above notation refers to a distribution that has the prescribed mean vectors and covariance matrices but are otherwise arbitrary. In the following, it can also be assumed that the mean vectors for the two classes are different.

An additional data vector z for which w^{T}z ≧b can be classified as belonging to the class associated with x_{+. Otherwise, the data vector can be classified as belonging to the class associated with x}_{-. Referring to relation (C-}4), the term 1−α represents an upper bound on a “worst-case” (e.g., maximum) misclassification error, and the classifiers serve to minimize this “worse-case” error.

It can be shown that a solution for w and b can be derived if:
w^{T}\hat{x}₋+k(α)\sqrt{w^{T}Γ₋w}≦b≦w^{T}\hat{x}₊−k(α)\sqrt{w^{T}Γ₃₀ w}, (C-5)
where
k(α)=\sqrt{α/(1−α)}. (C-6)

By eliminating b, the problem corresponds to minimizing over w a loss function represented as:

$${L}_{MPM} ( w , X , y ) = \frac{\sqrt{{w}^{T} {Γ}_{+} w} + \sqrt{{w}^{T} {Γ}_{-} w}}{\vert {w}^{T} ( {\hat{x}}_{+} - {\hat{x}}_{-} ) \vert} . \qquad ( C \text{-} 7 )$$

By homogeneity, the problem can be reduced to computing:

$$ϕ \text{:} = {min}_{w} {‖ {Γ}_{+}^{1 / 2} w ‖}_{2} + {‖ {Γ}_{-}^{1 / 2} w ‖}_{2} : {w}^{T} ( {\hat{x}}_{+} - {\hat{x}}_{-} ) = 1. \qquad ( C \text{-} 8 )$$

In the above relation, ∥ . . . ∥₂ denotes a l₂-norm of a vector expressed as a square-root of a sum of its squared components. Relation (C-8) can be interpreted as a second-order cone program and is amenable to efficient solutions using interior-point methods for conic programming, such as, for example, disclosed in S. P. Boyd and L. Vandenberghe, Convex Optimization (Prentice-Hall, 2003). If w* is optimized for the above problem, an optimized upper bound on the misclassification error can be represented as:

$$1 - {α}_{*} = \frac{{ϕ}^{2}}{1 + {ϕ}^{2}} . \qquad ( C \text{-} 9 )$$

Also, an optimized b* can be represented as:
b_{*}=w_{*}^{T}\hat{x}₊−(1/φ)\sqrt{w_{*}^{T}Γ₊w_{*}}=w_{*}^{T}\hat{x}₋+(1/φ)\sqrt{w_{*}^{T}Γ₋w_{*}}. (C-10)

Typically, a minimax probability machine uses empirical estimates for the mean vectors and covariance matrices for the two classes. Empirical estimates may be used with appropriate shrinkage factors to handle estimation errors. In some instances, a kernel version of a minimax probability machine may be derived. In particular, a linear kernel can be used by first forming a Gram matrix of data values. Additional discussion regarding the loss function L_{MPM} can be found in G. R. G. Lanckriet, L. El Ghaoui, C. Bhattacharyya, and M. I. Jordan, A Robust Minimax Approach to Classification, Journal of Machine Learning Research, 3:555-582 (2002).

Interval Matrix Model Formulation

In accordance with the interval matrix model, the constraints shown in relation (C-4) should hold irrespective of variation of gene expression data in χ. Again, uncertainty in gene expression data can be taken to be smoothed out when computing class means \hat{x}_{±}.

It can be shown that relation (C-5) holds for every matrix Z∈χ. Given that Γ_{±} represent empirical covariance matrices, then:

$${‖ {Γ}_{\pm } w ‖}_{2} = {c}_{\pm } \sqrt{{\sum }_{i ∈ {I}_{\pm }} {[ {w}^{T} ( {x}_{i} - {\hat{x}}_{\pm } ) ]}^{2}} , \qquad ( C \text{-} 11 )$$

where c_{±}=1/\sqrt{N_{±}}. This leads to the relation:
w^{T}\hat{x}₋+c₋k(α)c₋σ₋(w)≦b≦w^{T}\hat{x}₊−k(α)c₊σ₊(w), (C-12)
where

$${σ}_{\pm } ( w ) \text{:} = {max}_{Z ∈ χ} \sqrt{{\sum }_{i ∈ {I}_{\pm }} {[ {w}^{T} ( {z}_{i} - {\hat{x}}_{\pm } ) ]}^{2}} = \sqrt{{\sum }_{i ∈ {I}_{\pm }} {[ {w}^{T} ( {x}_{i} - {\hat{x}}_{\pm } ) + {σ}_{i}^{T} \vert w \vert ]}^{2}} , \qquad ( C \text{-} 13 )$$

and where \hat{x}_{±} can be assumed to be provided exactly. Maximizing a subject to relation (C-12) leads to:

$$ϕ \text{:} = {min}_{w} {c}_{+} {σ}_{+} ( w ) + {c}_{-} {σ}_{-} ( w ) : {w}^{T} ( {\hat{x}}_{+} - {\hat{x}}_{-} ) = 1 , \qquad ( C \text{-} 14 )$$

which, by homogeneity, corresponds to minimizing the loss function shown in relation (C-1).

If w* is optimized, an optimized lower bound on a “worst-case” misclassification error is φ/(1+φ²), while an optimized b is represented as:
b_{*}=w_{*}^{T}\hat{x}₊−(1/φ)c₋σ₋(w)=w_{*}^{T}\hat{x}₋+(1/φ)c₊σ₊(w). (C-15)

Upper Bound

In some instances, an approximation may be made by using an upper bound as follows:

$$\begin{matrix}{min}_{w} {c}_{+} \sqrt{{\sum }_{i ∈ {I}_{+}} {[ {w}^{T} ( {z}_{i} - {\hat{x}}_{+} ) ]}^{2}} + {c}_{-} \sqrt{{\sum }_{i ∈ {I}_{-}} {[ {w}^{T} ( {z}_{i} - {\hat{x}}_{-} ) ]}^{2}} + {σ}^{T} \vert w \vert : {w}^{T} ( {\hat{x}}_{+} - {\hat{x}}_{-} ) = 1 , \\ where & ( C \text{-} 16 ) \\ σ \text{:} = \frac{1}{\sqrt{{N}_{+}}} {\sum }_{i ∈ {I}_{+}} {σ}_{i} + \frac{1}{\sqrt{{N}_{-}}} {\sum }_{i ∈ {I}_{-}} {σ}_{i} & ( C \text{-} 17 )\end{matrix}$$

corresponds to a weighted sum of gene expression intervals with respect to the two classes.

Using the above approximation, it can be shown that the interval matrix model formulation leads to an l₁-norm regularization term. This regularization term tends to produce sparse classifiers. The computational cost of solving a problem with this regularization term is approximately the same as for the original formulation. For any particular loss function L, a “worse-case” methodology can be used to obtain values for w and b that reduce (e.g., minimize) a worse-case value of the loss function L over various possible realizations of the gene expression data. This “worse-case” methodology can be represented as:

$${min}_{w , b} {max}_{Z ∈ χ ( ρ )} L ( w , b , Z , y ) . \qquad ( 5 )$$

The function to be minimized in the above relation can be referred to as a “worse-case” loss function L^{wc}.

Advantageously, the “worse-case” loss function L^{wc} can inherit certain convexity properties of the loss function L (e.g., the loss function L_{SVM}, L_{LR}, or L_{MPM}), and the “worse-case” methodology can lead to a convex optimization problem that is amenable to efficient solutions using polynomial-time interior-point methods. Examples of polynomial-time interior-point methods include, for example, those arising in the context of linear programming, second-order cone programming (i.e., a generalization of linear programming that handles l₂-norm bounds), and constrained maximum entropy. Additional discussion regarding polynomial-time interior-point methods can be found in e.g., S. P. Boyd and L. Vandenberghe, “Convex Optimization” (Prentice-Hall, 2003); and Y. Nesterov and A. Nemirovsky, “Interior Point Polynomial Methods in Convex Programming: Theory and Applications” (SIAM, Philadelphia, Pa., 1994), the disclosures of which are incorporated herein by reference in their entireties.

The “worse-case” methodology described herein facilitates obtaining robust classifiers that account for uncertainty involved in real-world experiments. Advantageously, such robust classifiers reduce the possibility of misclassification errors that can arise when gene expression data are provided approximately in terms of intervals of confidence. For example, referring to FIG. 2, the “worse-case” methodology allows derivation of a linear classification function (e.g., the linear classification function 204) that adequately separates clusters of gene expression datasets even if gene expression levels can take different values within respective ranges of variation.

For certain applications, the “worse-case” methodology can be implemented using an approximation for the “worst-case” loss function L^{wc}. The approximation for the “worst-case” loss function L^{wc} is based on a l₁-norm regularization of the original loss function (e.g., the loss function L_{SVM}, L_{LR}, or L_{MPM}). In particular, the approximation for the “worst-case” loss function L^{wc} is based on an upper bound represented as:

$${L}^{wc} ( w , b ) = {max}_{Z ∈ χ} L ( w , b , Z , y ) \leq L ( w , b , X , y ) + {ρσ}^{T} \vert w \vert , \qquad ( 6 )$$

where |w| represents a vector having n components that are absolute values of corresponding components of the classifier vector w. σ is a vector with n non-negative components that are derived based on the matrix Σ. For cases where the original loss function is L_{SVM} or L_{LR}, the vector a can be represented as:

$$σ \text{:} = {\sum }_{i = 1}^{N} {σ}_{i } \qquad ( 7 )$$

where σᵢ represents an i^{th} column of the matrix Σ. Thus, for the loss functions L_{SVM} and L_{LR}, each component of the vector σ corresponds to a sum of gene expression intervals for a particular gene with respect to the N compounds. For cases where the original loss function is L_{MPM}, the vector σ can be represented as:

$$σ \text{:} = \frac{1}{\sqrt{{N}_{+}}} {\sum }_{i ∈ {I}_{+}} {σ}_{i} + \frac{1}{\sqrt{{N}_{-}}} {\sum }_{i ∈ {I}_{-}} {σ}_{i} \qquad ( 8 )$$

where σᵢ again represents an i^{th} column of the matrix Σ, and I₊ and I₋ represent indices associated with compounds in class+ and class−, respectively. Thus, for the loss function L_{MPM}, each component of the vector corresponds to σ weighted sum of gene expression intervals for a particular gene with respect to the two classes of compounds.

Additional discussion regarding the loss function L_{MPM} can be found in e.g., G. R. G. Lanckriet, L. El Ghaoui, C. Bhattacharyya, and M. I. Jordan, “A Robust Minimax Approach to Classification,” Journal of Machine Learning Research, 3:555-582 (2002), the disclosure of which is incorporated herein by reference in its entirety.

4. Extensions and Variations of the “Worse-Case” Methodology

Implementation Uncertainties

It is contemplated that a “worse-case” methodology can be applied to cases where one or more of gene expression data, the labeling vector y, and the set of classifiers are subject to uncertainty. In this section, application of the “worse-case” methodology to account for uncertainties (e.g., errors) in classifiers is discussed. The following discussion relates to an implementation for the loss function L_{SVM}. However, it should be recognized that a similar implementation can be used for the loss fluctions L_{LR} and L_{MPM}.

Specifically, the following optimization problem can be considered:

$${min}_{w , b} {max}_{‖ Δ w ‖ ∞ \leq δ} {L}_{SVM} ( w + Δ , b , X , y ) . \qquad ( D \text{-} 1 )$$

In the above relation, the matrix Z can be assumed to be fixed to its “typical” value given by the matrix X. Errors for w may be based on, for example, ignoring components having small magnitudes, and δ can represent an absolute measure of these errors. In this sense, a larger δ ensures that more components of w can be safely ignored, thus resulting in a sparser classifier vector.

A “worst-case” loss function can be represented as:

$${L}_{SVM}^{wc} = {\sum }_{i = 1}^{N} {( 1 - {y}_{i} ( {w}^{T} {z}_{i} + b ) + δ {‖ {x}_{i} ‖}_{1} )}_{+ ,} \qquad ( D \text{-} 2 )$$

which can be minimized as a linear program. In the above relation, ∥ . . . 81₁ denotes an l₁-norm of a vector expressed as a sum of absolute values of its components. δ can be viewed as a variable, and minimizing a combination of the above loss function and a function that decreases with δ leads to a trade-off between the number of “worse case” misclassification errors and sparsity of classifiers.

In some instances, ignoring components of w can be based on relative size rather than absolute size, such that δ can depend on w. For example, δ can be represented as δ= k∥w∥₁, where k>0 is fixed. A “worst-case” loss function can then be interpreted as of the type shown in relation (A-2), with ρ=k, and the matrix Σ including columns set to:
σᵢ=∥xᵢ∥₁. (D-3)

It is contemplated that the “worse-case” methodology can also account for both implementation errors and uncertainties in gene expression data. Specifically, the following optimization problem can be considered:

$${min}_{w , b} {max}_{‖ Δ w ‖ ∞ \leq δ , Z ∈ χ ( ρ )} {L}_{SVM} ( w + Δ , b , Z , y ) . \qquad ( D \text{-} 4 )$$

which can be represented as a linear program having Nn variables. An upper bound on a “worst-case” loss function can be obtained by maximizing over Δw independently in the linear and the norm term in relation (A-2), resulting in:

$${L}_{SVM}^{wc} ( w , b ) = {\sum }_{i = 1}^{N} {( 1 - {y}_{i} ( {w}^{T} {z}_{i} + b ) + {ρσ}_{i}^{T} ( \vert w \vert + δ 1 ) + δ {‖ {\hat{x}}_{i} ‖}_{1} )}_{+} . \qquad ( D \text{-} 5 )$$

Labeling Errors

In this section, application of a “worse-case” methodology to account for uncertainties in a labeling vector is discussed. It can be assumed that the matrix Z is fixed to its “typical” value given by the matrix X, while the labeling vector y (e.g., a ±1 labeling vector) is subject to uncertainty. It can also be assumed that k represents the number of components ofy subject to a change in sign, where 0<k<N. In some instances, k can be assumed to be fixed and can represent a bound on the number of labeling errors. The following discussion relates to an implementation for the loss function L_{SVM}. However, it should be recognized that a similar implementation can be used for the loss functions L_{LR} and L_{MPM}.

Specifically, the following optimization problem can be considered:

$$\begin{matrix}{min}_{w , b} {max}_{z ∈ 𝒴 ( y , k )} {L}_{SVM} ( w , b , X , z ) . \\ where & ( D \text{-} 6 ) \\ 𝒴 ( y , k ) = \{ z \text{:} {z}_{i} = ( 1 - 2 {δ}_{i} ) {y}_{i} , i = 1 , … , N , δ ∈ {[ 0 \; 1 ]}^{N} , {1}^{T} δ \leq k \} & ( D \text{-} 7 )\end{matrix}$$

represents a sign uncertainty in the labeling vector y.

To derive a solution to relations (D-6) and (D-7), a sub-problem can be first considered. In particular, given α and y∈R^{N} and k<N one can define:

$$ϕ = {max}_{z ∈ 𝒴 ( y , k )} {\sum }_{i = 1}^{N} {( 1 - {α}_{i} {z}_{i} )}_{+} . \qquad ( D \text{-} 8 )$$

Using relation (D-8), it can be shown that:

$$\begin{matrix}\begin{matrix}ϕ = {max}_{0 \leq t \leq 1} {max}_{z ∈ 𝒴} {\sum }_{i = 1}^{N} {t}_{i} ( 1 - {α}_{i} {z}_{i} ) \\ = {max}_{0 \leq t \leq 1} {max}_{δ ∈ Δ} {\sum }_{i = 1}^{N} ( {t}_{i} ( 1 - {α}_{i} {y}_{i} ) + 2 {δ}_{i} {t}_{i} {y}_{i} {α}_{i} ) \\ = {max}_{0 \leq t \leq 1} ( {1}^{T} ( t - α ( y , t ) ) + {max}_{δ ∈ Δ} {δ}^{T} α ( y , t ) ) ,\end{matrix} \\ where & ( D \text{-} 9 ) \\ Δ = \{ δ ∈ {[ 0 \; 1 ]}^{N} \text{:} {1}^{T} δ \leq k \} , and {( α ( y , t ) )}_{i} = {t}_{i} {y}_{i} {α}_{i} , i = 1 , … , N . & ( D \text{-} 10 )\end{matrix}$$

Using duality of a linear program, it can be shown that for a particular t:

$${max}_{δ ∈ Δ} {δ}^{T} α ( y , t ) = {min}_{λ \geq 0} λ k + {1}^{T} {( α ( y , t ) - λ )}_{+} . \qquad ( D \text{-} 11 )$$

Hence, the sub-problem can be represented as:

$$ϕ = {max}_{0 \leq t \leq 1} {min}_{λ \geq 0} {\sum }_{i = 1}^{N} ( {t}_{i} ( 1 - {α}_{i} {y}_{i} ) + λ k + {( {α}_{i} {y}_{i} {t}_{i} - λ )}_{+} ) . \qquad ( D \text{-} 12 )$$

Using duality again, it can be shown that:

$$\begin{matrix}ϕ = {min}_{λ \geq 0} {max}_{0 \leq t \leq 1} {\sum }_{i = 1}^{N} ( {t}_{i} ( 1 - {α}_{i} {y}_{i} ) + λ k + {( {α}_{i} {y}_{i} {t}_{i} - λ )}_{+} ) \\ = {min}_{λ \geq 0} {\sum }_{i = 1}^{N} {max}_{ 0 \leq u \leq 1} ( u ( 1 - {α}_{i} {y}_{i} ) + λ k + {( {α}_{i} {y}_{i} u - λ )}_{+} ) \\ = {min}_{λ \geq 0} {\sum }_{i = 1}^{N} {max}_{ u = 0 , 1} ( u ( 1 - {α}_{i} {y}_{i} ) + λ k + {( {α}_{i} {y}_{i} u - λ )}_{+} ) \\ = {min}_{λ \geq 0} λ kN + {\sum }_{i = 1}^{N} {( ( 1 - {α}_{i} {y}_{i} ) + {( {α}_{i} {y}_{i} - λ )}_{+} )}_{+} .\end{matrix} \qquad ( D \text{-} 13 )$$

Accordingly, relation (D-6) can be represented as:

$${min}_{λ \geq 0 , w , b} λ kN + {\sum }_{i = 1}^{N} {( ( 1 - {y}_{i} ( {w}^{T} {x}_{i} + b ) ) + {( {y}_{i} ( {w}^{T} {x}_{i} + b ) - λ )}_{+} )}_{+} \qquad ( D \text{-} 14 )$$

which can be solved as a linear program. It should be recognized that the original problem is recovered if k=0, namely, there are no labeling errors.

Ellipsoidal Uncertainty Models

It is contemplated that uncertainties in gene expression data can be represented using various uncertainty models. This section discusses application of a “worse-case” methodology to the case where χ is represented as a product of ellipsoids, namely
χ={Z=[z₁, . . . , z_{N}]∈R^{N×n}:(zᵢ−xᵢ)^{T}Γᵢ⁻¹(zᵢ−xᵢ)≦1, i=1, . . . , N} (D-15)
where positive-definite matrices Γᵢ (i=1, . . . , N) represent uncertainties in gene expression data.

Using this ellipsoidal uncertainty model for a support vector machine, a “worst-case” loss function can be represented as:

$${L}_{SVM}^{wc} ( w , b ) = {\sum }_{i = 1}^{N} {( 1 - {y}_{i} ( {w}^{T} {x}_{i} + b ) + {‖ {Γ}_{i}^{1 / 2} w ‖}_{2} )}_{+} , \qquad ( D \text{-} 16 )$$

where ∥ . . . ∥₂ denotes an l₂-norm of a vector expressed as a sum of squared absolute values of its components. An upper bound for the “worst-case” loss function can be represented as:

$${L}_{SVM}^{wc} ( w , b ) = {\sum }_{i = 1}^{N} ( {( 1 - {y}_{i} ( {w}^{T} {x}_{i} + b ) )}_{+} + {‖ {Γ}_{i}^{1 / 2} w ‖}_{2} ) . \qquad ( D \text{-} 17 )$$

Application of the “worse-case” methodology to relations (D-16) and (D-17) leads to a second-order cone program, which can be solved using interior-point methods for conic programming, such as, for example, disclosed in S. P. Boyd and L. Vandenberghe, Convex Optimization (Prentice-Hall, 2003). The upper bound shown in relation (D-17) is associated with a regularization of the original loss function, such that regularization parameters depend on characteristics of the ellipsoidal uncertainty model. The ellipsoidal uncertainty model relates to a sum of 1₂-norms. In some instances, solutions can be derived by assuming that the matrices Γᵢ are equal.

Application of the “worse-case” methodology to relations (D-16) and (D-17) can be interpreted in terms of classification of ellipsoids. When the matrices Γᵢ are set to multiples of identity, the problem reduces to a variation of a conventional support vector machine with an l₂-norm term instead of a squared l₂-norm term.

Using the ellipsoidal uncertainty model for logistic regression, a “worst-case” loss fuinction can be represented as:

$${L}_{LR}^{wc} ( w , b ) = {\sum }_{i = 1}^{N} ( log ( 1 + {ⅇ}^{{w}^{T} {x}_{i} + b + ( 1 - {2}_{{y}_{i}} ) {‖ {Γ}_{i}^{1 / 2} w ‖}_{2}} - {y}_{i} ( {w}^{T} {x}_{i} + b - {‖ {Γ}_{i}^{1 / 2} w ‖}_{2} ) ) , \qquad ( D \text{-} 18 )$$

which is convex in w and b and is amenable to efficient solutions using interior-point methods, such as, for example, methods implemented in MOSEK Optimization Software.

And, using the ellipsoidal uncertainty model for a minimax probability machine, a “worst-case” loss function can be represented as:

$${L}_{MPM}^{wc} ( w , b ) = {c}_{+} \sqrt{{\sum }_{i ∈ {l}_{+}} {[ {w}^{T} ( {x}_{i} - {\hat{x}}_{+} ) + {‖ {Γ}_{i}^{1 / 2} w ‖}_{2} ]}^{2}} + {c}_{-} \sqrt{{\sum }_{i ∈ {l}_{-}} {[ {w}^{T} ( {x}_{i} - {\hat{x}}_{-} ) + {‖ {Γ}_{i}^{1 / 2} w ‖}_{2} ]}^{2}} , \qquad ( D \text{-} 19 )$$

which leads to a second-order cone program that can be solved using interior-point methods for conic programming. An upper bound for the above loss function also relates to a sum of l₂-norms, as discussed above for a support vector machine.

5. Numerical Implementation of “Worse Case” Methodology

In this section, additional details regarding numerical implementation of a “worse-case” methodology to the loss functions L_{SVM}, L_{LR}, and L_{MPM} are provided. For example, the “worse-case” methodology can be implemented using conventional convex optimization software, such as, for example, MOSEK Optimization Software.

Basic Implementation of “Worse Case”

For a support vector machine, the “worse-case” methodology represented by relation (A-3) can be implemented as a linear programming problem:

$$\begin{matrix}{min}_{{w}_{n} , {w}_{p} , b , z} {z}^{T} v \text{:} & {y}_{i} ( {( {w}_{p} - {w}_{n} )}^{T} {\hat{x}}_{i} + b ) \geq 1 - {z}_{i} + {ρσ}_{i}^{T} ( {w}_{p} + {w}_{n} ) , \\ & {z}_{i} \geq 0 , i = 1 , … , N , \\ & {w}_{p} \geq 0 , {w}_{n} \geq 0 ,\end{matrix} \qquad ( E \text{-} 1 )$$

where w=wₚ−wₙ, and vᵢ=N/n_{±} if i∈I_{±}. The vector v allows misclassification errors for the two classes to be weighted differently, such as, for example, by penalizing errors more for a smaller class. Relation (E-1) has 2n+N+1 variables and N constraints (without counting sign constraints on the variables themselves), which can be handled separately by MOSEK Optimization Software.

The l₁-norm regularization problem shown in relation (A-6) can be implemented as:

$$\begin{matrix}{min}_{{w}_{n} , {w}_{p} , b , z} {z}^{T} v + {ρσ}^{T} ( {w}_{p} + {w}_{n} ) \text{:} & \begin{matrix}{y}_{i} ( {( {w}_{p} - {w}_{n} )}^{T} {\hat{x}}_{i} + b ) \geq 1 - {z}_{i} , \\ {z}_{i} \geq 0 , i = 1 , … , N ,\end{matrix} \\ & {w}_{p} \geq 0 , {w}_{n} \geq 0 ,\end{matrix} \qquad ( E \text{-} 2 )$$

where w and v represent quantities as defined for relation (E-1), and cr represents a quantity as shown in relation (A-5). Relation (E-2) has the same number of variables and constraints as relation (E-1).

For logistic regression, the “worse-case” methodology represented by relation (B-13) can be implemented as a maximum entropy problem:

$$\begin{matrix}ψ = {min}_{λ , μ} {λ}^{T} log λ + {μ}^{T} log μ \text{:} & λ + μ = 1 , λ \geq 0 , {λ}^{T} 1 = {N}_{+} , \\ & \begin{matrix}[ {X}_{+} + ρ {\sum }_{+} , {X}_{-} - ρ {\sum }_{-} ] \\ λ \leq {X}_{+} 1 + ρ {\sum }_{+} 1\end{matrix} \\ & \begin{matrix}{X}_{+} 1 - ρ {\sum }_{+} 1 \leq \\ [ {X}_{+} - ρ {\sum }_{+} , {X}_{-} + ρ {\sum }_{-} ] λ .\end{matrix}\end{matrix} \qquad ( E \text{-} 3 )$$

Relation (E-3) has 2N variables and 2n+N+1 linear inequality constraints.

The problem shown in relation (B-23) can be implemented as:

$$\begin{matrix}ψ = {min}_{λ , μ} {λ}^{T} log λ + {μ}^{T} log μ \text{:} & λ + μ = 1 , λ \geq 0 , {λ}^{T} 1 = {N}_{+} , \\ & {X}_{+} 1 - ρσ \leq X λ \leq {X}_{+} 1 + ρσ ,\end{matrix} \qquad ( E \text{-} 4 )$$

where σ represents a quantity as shown in relation (B-20).

For a minimax probability machine, the “worse-case” methodology represented by elation (C-14) can be implemented as a second-order cone program:

$$\begin{matrix}{min}_{{w}_{p} , {w}_{n} , {t}_{\pm } , {u}_{\pm } , {s}_{\pm }} {c}_{+} {t}_{+} + {c}_{-} {t}_{-} \text{:} & {t}_{\pm } \geq {‖ {u}_{\pm } ‖}_{2} , \\ & \begin{matrix}{u}_{\pm } = {( {X}_{\pm } + ρ {\sum }_{\pm } )}^{T} {w}_{p} - \\ {( {X}_{\pm } - ρ {\sum }_{\pm } )}^{T} {w}_{n} - {s}_{\pm } 1 ,\end{matrix} \\ & {s}_{\pm } = {\hat{x}}_{\pm }^{T} ( {w}_{p} - {w}_{n} ) , {s}_{+} - {s}_{-} = 1 , \\ & {w}_{p} \geq 0 , {w}_{n} \geq 0 ,\end{matrix} \qquad ( E \text{-} 5 )$$

which has 2n+2N+4 variables and N+3 equality constraints.

And, relation (C-16) can be implemented as:

$$\begin{matrix}\begin{matrix}{min}_{ {w}_{ p} , {w}_{ n} , {t}_{\pm } \cdot {u}_{\pm } , {s}_{\pm }} \\ {c}_{+} {t}_{+} + c\_t \_ + {σ}^{T} ( {w}_{p} + {w}_{n} )\end{matrix} \text{:} {t}_{\pm } \geq {‖ {u}_{\pm } ‖}_{2} , & \\ {u}_{\pm } = {X}_{\pm }^{T} {w}_{p} - {X}_{\pm }^{T} {w}_{n} - {s}_{\pm } 1 , & \\ {s}_{\pm } = {\hat{x}}_{\pm }^{T} ( {w}_{p} - {w}_{n} ) , & {s}_{+} - {s}_{-} = 1 \\ {w}_{p} \geq 0 , & {w}_{n} \geq 0.\end{matrix} \qquad ( E \text{-} 6 )$$

where σ represents a quantity as shown in relation (C-17).

Implementation “Worse Case ” for Exploiting Sparsity

In some instances, a “worse-case” methodology can be implemented to exploit sparsity of one, or both, of the matrices X and Σ. For example, X may include various data values having small magnitudes, and one or more of these data values can be set to zero according to a filtering rule. An example of a filtering rule is to set X(i, j) to zero according to the criterion:
|X(i, j)|≦∈Σ(i, j), (E-7)
where ε represents a parameter (e.g., an adjustable parameter) that sets a threshold level. Using relation (E-7), the matrix X can be filtered such that it remains in the uncertainty set χ(ρ) for ε≦ρ. As a result, misclassification errors for a filtered training set can be the same as for an unfiltered training set. However, in some instances, misclassification errors for an additional set (e.g., a test set) can be different.

Sparsity of the matrix X can be exploited in the l₁-norm regularization problems discussed previously. For certain applications, the matrix Σ is typically not sparse. However, sparsity of the matrix X can be exploited by considering potential regularity of the matrix Σ. Regularity of a matrix refers to a condition where various data values of the matrix are the same or substantially the same. In particular, a regular matrix can be a rank-one modification of a sparse matrix. For example, it can be assumed that the matrix Σ is represented as:
Σ=σ_{avg}1^{T}+δΣ, (E-8)
where σ_{avg}>0 can be interpreted as an average of gene expression intervals with respect to the N compounds, and δΣ is a sparse matrix. It should be recognized that sparsity can be a special case of regularity with σ_{avg}=0.

When X is sparse and Σ is regular, a modification can be introduced to the implementations shown in relations (E-1), (E-3), and (E-5) to exploit both properties. For relation (E-1), for example, the modification can involve introduction of a new variable u and an associated constraint u≧σ_{avg}|w| to obtain:

$$\begin{matrix}{min}_{{w}_{n} , {w}_{p} , b , z , u} {z}^{T} υ \text{:} {y}_{i} ( {( {w}_{p} - {w}_{n} )}^{T} {\hat{x}}_{i} + b ) \geq 1 - {z}_{i} + & & \\ ρ u + {( ρ ( {δσ}_{ i} ) )}^{T} ( {w}_{ p} + {w}_{ n} ) , & & \\ {z}_{ i} >= 0 , i = 1 , … , N , & & \\ {w}_{ p} \geq 0 , \begin{matrix} {w}_{ n} \geq 0 , & u \geq {σ}_{ avg}^{ T} ( {w}_{ p} + {w}_{ n} )\end{matrix} & & \end{matrix} \qquad ( E \text{-} 9 )$$

Relation (E-9) can be interpreted as adding one column and one row to the original constraint matrix while preserving its sparsity. Similar results can be derived for logistic regression and minimax probability machine. It is contemplated that the “worse-case” methodology can also exploit situations where X is not sparse but regular.

As described above, using the approximation for the “worse-case” loss function L^{wc}, the “worse-case” methodology can be used to obtain values for w and b that reduce (e.g., minimize) the “worse-case” loss function LWC. In accordance with this approximation, the “worse-case” methodology can naturally lead to sparse classifiers, such that various classifiers (e.g., a majority or a vast majority of classifiers) included in w and b have zero or relatively small magnitudes and can be omitted from further analysis without substantial loss of information. Accordingly, robustness and sparsity in the classifiers can both be achieved in accordance with the “worse-case” methodology.

Sparse classifiers are typically associated with a linear classification function that is substantially aligned with various dimensions in a multi-dimensional space. For example, referring to FIG. 2, the “worse-case” methodology allows derivation of a linear classification function (e.g., the linear classification function 204) that is substantially aligned with an axis associated with gene 2. Sparse classifiers are particularly desirable when n is a large number, since computational efficiency is improved by allowing certain genes to be ignored when classifying gene expression data. Moreover, sparse classifiers facilitate interpretation of results by, for example, allowing identification of a subset of genes that may play a greater role in a biological activity or a biological state. This subset of genes can be identified by, for example, sorting the classifiers based on magnitude and identifying genes associated with classifiers having a greater magnitude. Additional methods for generating sparse classifiers based on reduced subsets of high impact genes are described in U.S. patent application Ser. No. 60/579,183 filed Jun. 10, 2004 (entitled, “Sufficient and Necessary Gene Signatures”) which is hereby incorporated by reference herein for all purposes.

Cross- Validation of Classifiers

Cross-validation of a classifier or signature's performance is an important step for determining whether the performance of the classifier is adequate. Cross-validation may be carried out by first randomly splitting the full dataset (e.g. a 60/40 split). A training classifier is derived from the training set composed of 60% of the samples and used to classify both the training set and the remaining 40% of the data, referred to herein as the test set. In addition, a complete classifier is derived using all the data. The performance of these classifiers may be measured in terms of log odds ratio (LOR) or the error rate (ER) defined as:
LOR=ln(((TP+0.5)*(TN+0.5))/((FP+0.5)*(FN+0. 5)))
and
ER=(FP+FN)/N;

where TP, TN, FP, FN, and N are true positives, true negatives, false positives, false negatives, and total number of samples to classify, respectively, summed across all the cross validation trials. The performance measures are used to characterize the complete classifier, the average of the training or the average of the test signatures.

The loss function algorithms described above are capable of generating a plurality of different classification rules each with varying degrees of performance for the classification task. In order to identify the classifiers that are to be considered “valid,” a threshold performance is selected for the particular classification question. In one preferred embodiment, the classifier threshold performance is set as log odds ratio greater than or equal to 4.00 (i.e. LOR≧4.00). However, higher or lower thresholds may be used depending on the particular dataset and the desired properties of the signatures that are obtained.

Using the methods described herein, two or more valid classifiers may be generated that are redundant or synonymous for a variety of reasons. Different classification questions (i.e. class definitions) may result in identical classes and therefore identical classifiers. For instance, the following two class definitions define the exact same compounds in a chemogenomic database based on gene expression data: (1) all treatments with molecules structurally related to statins; and (2) all treatments with molecules having an IC₅₀<1 μM for inhibition of the enzyme HMG CoA reductase.

In addition, when a large dataset is queried with the same classification question using different loss function algorithms (or even the same algorithm under slightly different conditions) different, valid classifiers may be obtained. These different classifiers may or may not comprise overlapping sets of variables; however, they each can accurately identify members of the class of interest.

It should be recognized that the embodiments discussed above are provided by way of example, and various other embodiments are contemplated. For example, while certain embodiments have been described in connection with classifying biological gene expression data, it should be recognized that the methodology described herein can be applied to other types of biological data, or to any multi-dimensional dataset.

One of ordinary skill will recognize that the methods of the present invention may be applied to multivariate data in physical science applications such as climate prediction, or oceanography, where large datasets are acquired and linear classification is a useful method of analysis.

Large dataset classification problems also are common in the finance industry (e.g. banks, insurance companies, stock brokers, etc.) A typical finance industry classification question is whether to grant a new insurance policy (or home mortgage) versus not. The variables to consider are any information available on the prospective customer or, in the case of stock, any information on the specific company or even the general state of the market. The finance industry equivalent to a “Group signature” would be financial signatures for a specific decision. The present invention would allow one to generate a classifier for a particular financial analysis question from a large set of financial data.

Also, while certain embodiments have been described in connection with a binary classification rule, it should be recognized that the methodology described herein can also be applied in connection with a multi-class classification rule. In addition, it should be recognized that the methodology described herein can be applied to various other types of loss functions.

As another example, some embodiments of the invention relate to deriving Group Signatures in accordance with the methodology described herein. In some instances, a Group Signature can be derived by sorting classifiers based on magnitude and identifying a subset of genes associated with classifiers having a greater magnitude. Advantageously, the methodology described herein can naturally lead to sparse classifiers, which allow for “short” Group Signatures (e.g., Group Signatures that indicate relatively small subsets of genes). A Group Signature is useful for identifying gene regulatory pathways most affected by a set of stimuli (e.g., a class of compounds) and, by extension, a subset of genes most involved in responding to the set of stimuli. A Group Signature is also useful for characterizing a new stimulus and for predicting a biological activity of the stimulus. In some instances, a database of Group Signatures for various classes of compounds (e.g., a fibrate Group Signature, an ACE inhibitor Group Signature, a caspase inhibitor Group Signature, and the like) can be compiled, where each Group Signature indicates, for example, 10 to 20 genes. The resulting Group Signature database can be substantially smaller than a typical database of gene expression data and can be queried rapidly. Group Signatures can also be derived in accordance with the methods disclosed in the co-pending and co-owned patent application to Natsoulis, entitled “Drug Signatures,” U.S. application Ser. No. 10/378,002, filed Feb. 28, 2003, the disclosure of which is incorporated herein by reference in its entirety.

Classification Rules Useful for Diaenostic Development

Classification rules (i.e. classifiers or signatures) provided by the methods of the present invention may be used in the development of devices for analytical measurements (e.g. diagnostic devices). For example, a Group Signature comprising a sparse linear classifier made by the methods of the present invention may be “embodied” in a set of analytical reagents (e.g. sequence specific polynucleotide probes, or antigen specific antibodies). These reagents may be immobilized to create a solid phase device (e.g. a polynucleotide array), or used in a solution phase assay (e.g. RT-PCR). For example, one or more solid supports may be provided with various regions, and each region can include polynucleotides capable of specifically binding sequences that make up a particular Group Signature. Thus, a Group Signature chip may have a first region containing probes specific for a fibrate Group Signature, a second region containing probes specific for a phenyl-acetic acid (e.g., aspirin, naproxen, and ibuprofen) Group Signature, and so forth. The probes for each Group Signature can be selected so that they do not overlap or so that they overlap to a minimal degree. Alternatively, if two or more Group Signatures indicate a common set of genes, the chip can be arranged to include probes for the common set as the intersection between two Group Signatures.

Methods of using classifiers for a large multivariate dataset to develop diagnostic devices are described in detail in U.S. patent application Ser. No. 60/565,793 filed Apr. 26, 2004 (entitled “Reduced Subsets of Multivariate Data Useful for Diagnostic Development”) which is hereby incorporated by reference herein for all purposes.

ComDuter-based Embodiments of the Invention

The methods for classifying multi-dimensional biological datasets provided by the present invention may be embodied in computer-based products such as computer-executable code stored in a computer-readable medium. Any of a wide variety of common computer systems well-known in the art, and typically including one or more computers, may be used to store, retrieve, and analyze the biological dataset information and derive classification rules using the methods and algorithms disclosed herein. Computer systems useful with the present invention may be as simple as a stand-alone computer having a form of data storage (e.g., a computer-readable medium). Alternatively, the computer system can include a network including two or more computers linked together via, for example, a server. The network can include an Intranet, an Internet connection, or both. In some instances, the computer systems are provided with processors and software for receiving and storing gene expression data or any other multi-dimensional biological data in a database and for executing operations on the stored data. The computer systems can be linked to databases such as Genbank and DrugMatrix (Iconix Pharmaceuticals, Inc., Mountain View, Calif.).

The present invention also provides a computer storage product including a computer-readable medium having computer-executable code thereon for performing various computer-implemented operations used to derive classification rules. Examples of computer-executable code include machine code, such as produced by a compiler, and files containing higher-level code that are executed by a computer using an interpreter. Source code may be implemented using Java, C++, other object-oriented programming language and development tools, or a higher-level mathematical language such as Matlab® (The Mathworks Inc., Natick, Mass.). For example, the “worse-case” methodology for deriving a classifier, as described by the mathematical framework disclosed herein, may be coded and implemented as an executable program using Matlab® (The Mathworks Inc., Natick, Mass.) by those of ordinary skill in the computer programming arts.

Additional examples of computer-executable code include encrypted code and compressed code. The term “computer-readable medium” is used herein to include any medium that is capable of storing or encoding a sequence of instructions or codes for performing the methods described herein. The media and code may be those specially designed and constructed for the purposes of the invention, or they may be of the kind well known and available to those having ordinary skill in the computer software arts. Examples of computer-readable media include, but are not limited to: magnetic media such as hard disks, floppy disks, and magnetic tape; optical media such as CD-ROMs and holographic devices; magneto-optical media such as floptical disks; carrier waves signals; and hardware devices that are specially configured to store and execute program code, such as application-specific integrated circuits (“ASICs”), programmable logic devices (“PLDs”), read only memories (“ROMs”), random access memories (“RAMs”), erasable programmable read only memories (“EPROMs”), and electrically erasable programmable read only memories (“EEPROMs”).

Moreover, some embodiments of the invention may be downloaded as a computer program product, where the program may be transferred from a remote computer (e.g., a server) to a requesting computer (e.g., a client) by way of data signals embodied in a carrier wave or other propagation medium via a communication link (e.g., a modem or network connection). Accordingly, as used herein, a carrier wave can be regarded as a computer-readable medium.

Other embodiments of the invention may be implemented in hardwired circuitry in place of, or in combination with, machine-executable software instructions.

### EXAMPLES

The following example is provided as a guide for the practitioner of ordinary skill in the art. The example should not be construed as limiting the invention, as the example merely provides specific methodology useful in understanding and practicing the invention.

### Example 1

Construction of Reference Gene Expression Dataset

In vivo short-term repeat dose rat studies were conducted on over 580 test compounds, including marketed and withdrawn drugs, environmental and industrial toxicants, and standard biochemical reagents. The data from these in vivo experiments was used to form the basis of a comprehensive chemogenomic reference database (“DrugMatrix™”) that also includes data from the clinical chemistry and hematology experiments and information extracted from the literature. The construction of this database is described in U.S. application Ser. No. 10/854,609 filed May 24, 2004, which is hereby incorporated by reference for all purposes. This chemogenomic reference database was used in the following Example to provide the expression dataset from which classification functions were derived according to the various loss finctions.

Briefly, rats (three per group) were dosed daily at either a low or high dose. The low dose was an efficacious dose estimated from the literature and the high dose was an empirically-determined maximum tolerated dose, defined as the dose that causes a 50% decrease in body weight gain relative to controls during the course of the 5 day range finding study. Animals were necropsied on days 0.25, 1, 3, and 5 or 7. Up to 13 tissues (e.g., liver, kidney, heart, bone marrow, blood, spleen, brain, intestine, glandular and nonglandular stomach, lung, muscle, and gonads) were collected for histopathological evaluation and microarray expression profiling on the Amersham CodeLink™ RU1 platform. In addition, a clinical pathology panel consisting of 37 clinical chemistry and hematology parameters was generated from blood samples collected on days 3 and 5.

### Example 2

Classification of Gene Expression Data Using Various Loss Functions

Numerical experiments were performed on data from a chemogenomic gene expression dataset made according to Example 1. The objective of the numerical experiments was to derive sparse classifiers (i.e. classifiers comprising a relatively small number of genes) that were useful for distinguishing three particular classes of compounds from other compounds with good performance. The three compound classes for which classifiers were derived are: fibrates, statins and azoles.

The gene expression data was assembled into a training set based on a matrix Xand a matrix Σ (i.e. matrices of the type described in FIG. 1). The matrix X included logarithm of ratios of gene expression levels relative to baseline gene expression levels for n=8565 genes and N=194 compounds. The matrix Σ included standard deviations associated with 3 measurements for each compound.

Three different labeling vectors were used in connection with classification of each particular class of compounds with respect to all other compounds. The fibrate class of compounds included N₊=36 compounds, the statin class of compounds included N₊=31 compounds, and the azole class of compounds included N₊=54 compounds. In the numerical experiments, a 3/2 training set to test set ratio was used, and compounds associated with the test set were used to evaluate average test set error rates (i.e., number of misclassification errors divided by the number of compounds in the test set) as well as average classifier lengths (i.e., number of non-zero components of classifiers). The average test set error rates and average classifier lengths were obtained using 10-fold cross-validation, and the results were averaged again over the three classification tasks of “fibrates versus other compounds,” “statins versus other compounds,” and “azoles versus other compounds.”

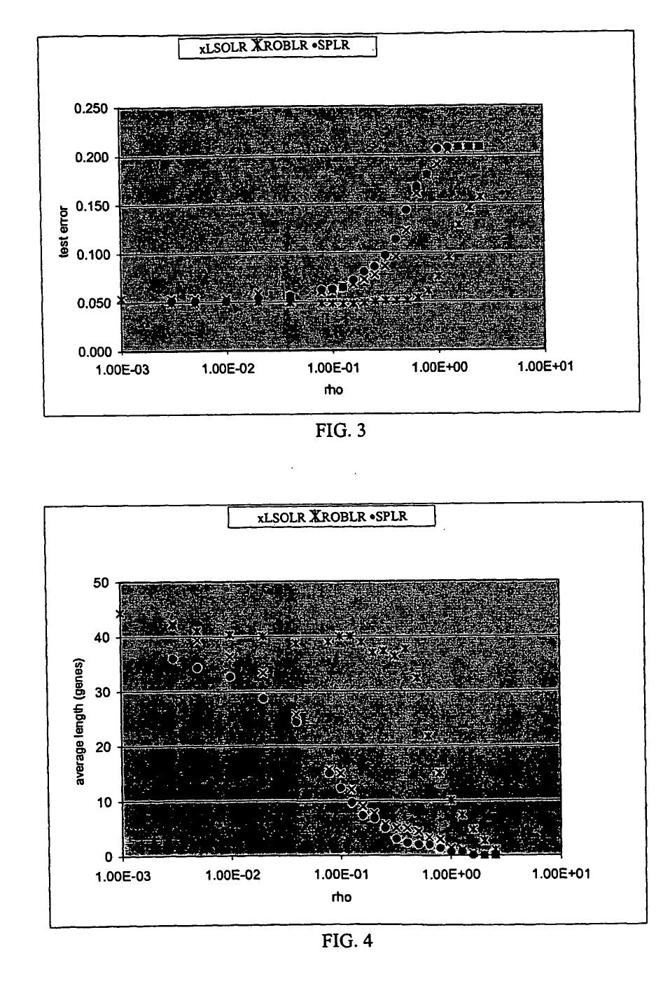

FIG. 3 and FIG. 4 illustrate results of the numerical experiments for the logistic regression loss function L_{LR}. Referring to FIG. 3 and FIG. 4, the labels “ROBLR” and “SPLR” represent implementations of the “worse-case” methodology using the loss function L_{LR} and its associated upper bound approximation, respectively, while the label “LSOLR” represent an implementation of a conventional approach based on “LSO regularization.” Here, results for the “LSOLR” implementation were obtained using relation (6) with all components of a set to 1.

FIG. 3 illustrates performance of the “LSOLR,” “ROBLR,” and “SPLR” implementations for the three classification tasks discussed previously. Here, performance is shown as a function of the parameter ρ (labeled as “rho”) and is measured based on average test set error rates. As illustrated in FIG. 3, the “LSOLR,” “ROBLR,” and “SPLR” implementations exhibited a similar level of performance for certain values of ρ, although performance was observed to be generally better for the “ROBLR” implementation. In particular, this improved performance for the “ROBLR” implementation was observed for various values of ρ, and differences in average test set error rates were observed even at relatively small values of ρ. For example, for ρ=10⁻⁶ (not illustrated in FIG. 3), the “ROBLR” implementation exhibited an average test set error rate of about 0.041 (or about 4.1%), the “SPLR” implementation exhibited an average test set error rate of about 0.047 (or about 4.7%), and the “LSOLR” implementation exhibited an average test set error rate of about 0.051 (or about 5.1%). The “LSOLR” implementation could achieve a performance similar to that of the “ROBLI” and “SPLR” implementations for certain values of ρ, but, as discussed below, the “LSOLR” implementation resulted in longer average classifier lengths.

Turning next to FIG. 4, average classifier lengths obtained for the “LSOLR,” “ROBLR,” and “SPLR” implementations for the three classification tasks are illustrated. Here, average classifier lengths are shown as a function of the parameter ρ (labeled as “rho”). For a similar level of performance, the “LSOLR” implementation was observed to produce longer average classifier lengths than the “SPLR” implementation (e.g., up to about 4 times longer for certain values of ρ). For example, for ρ=10⁻⁶ (not illustrated in FIG. 4), the “ROBLR” implementation produced an average classifier length of about 61, the “SPLR” implementation produced an average classifier length of about 40, and the “LSOLR” implementation produced an average classifier length of about 46. The “ROBLR” implementation achieved the best performance with respect to the three implementations but produced average classifier lengths that are somewhat longer than that of the “SPLR” implementation. The numerical experiments indicate that one advantage of the “worst-case” methodology using the “SPLR” implementation is to obtain a better or similar level of performance while using a smaller number of genes, which can be desirable to facilitate interpretation of the results.

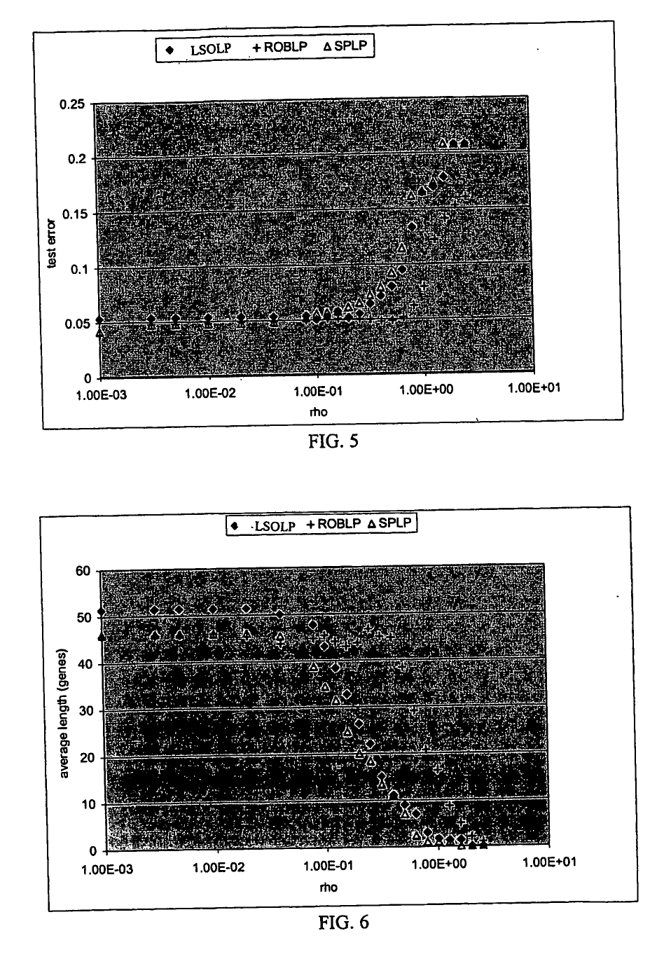

FIG. 5 and FIG. 6 illustrate results of the numerical experiments for the support vector machine loss function L_{SVM}. Referring to FIG. 5 and FIG. 6, the labels “ROBLP” and “SPLP” represent implementations of the “worse-case” methodology using the loss function L_{SVM} and its associated upper bound approximation, respectively, while the label “LSOLP” represent an implementation of a conventional approach based on “LSO regularization.” Here, results for the “LSOLP” implementation were obtained using relation (6) with all components of σ set to 1.

FIG. 5 illustrates performance of the “LSOLP,” “ROBLP,” and “SPLP” implementations for the three classification tasks as a function of the parameter ρ (labeled as “rho”). FIG. 6 illustrates average classifier lengths obtained for the “LSOLP,” “ROBLP,” and “SPLP” implementations for the three classification tasks as a function of the parameter ρ. Similar results were obtained for the loss function L_{SVM} as for the loss function L_{LR}. Thus, for example, performance was observed to be generally better for the “ROBLP” implementation. In particular, this improved performance for the “ROBLP” implementation was observed for various values of ρ, and differences in average test set error rates were observed even at relatively small values of ρ. For a similar level of performance, the “LSOLP” implementation was observed to produce longer average classifier lengths than the “SPLP” implementation. The numerical experiments indicate that the “worst-case” methodology can provide significant advantages across various types of loss functions. Overall, the “SPLP” implementation was observed to produce the best compromise in terms of performance, average classifier length, and computational time.

Table 1 describes two equally performing sparse linear classifiers (LOR=˜7.0) for the fibrate class of compounds that were generated by querying the same reference gene expression dataset (Example 1) with two different loss function algorithms: SPLP and SPLR. The genes comprising the variables in the classifiers are designated by their accession number and a brief description. The weights associated with each gene are also indicated. Each signature was trained on the exact same 60% of the multivariate dataset and then cross validated on the exact same remaining 40% of the dataset. Both signatures were found to exhibit the exact same level of performance as classifiers: two errors on the cross validation data set. The SPLP derived signature consists of 20 genes. The SPLR derived signature consists of eight genes. Only three of the genes from the SPLP signature are present in the eight gene SPLR signature.

TABLE 1
Two Gene Signatures for the Fibrate Class of Drugs

| | Accession | Weight | Unigene name |
|---|---|---|---|
| RLPC | K03249 | 1.1572 | enoyl-Co A, hydratase/3-hydroxyacyl |
| | | | Co A dehydrogenase |
| | AW916833 | 1.0876 | hypothetical protein RMT-7 |
| | BF387347 | 0.4769 | ESTs |
| | BF282712 | 0.4634 | ESTs |
| | AF034577 | 0.3684 | pyruvate dehydrogenate kinase 4 |
| | NM_019292 | 0.3107 | carbonic anhydrase 3 |
| | Al179988 | 0.2735 | ectodermal-neural cortex (with |
| | | | BTB-like domain) |
| | Al715955 | 0.211 | Stac protein (SRC homology 3 and |
| | | | cysteine-rich domain protein) |
| | BE110695 | 0.2026 | activating transcription factor 1 |
| | J03752 | 0.0953 | microsomal glutathione |
| | | | S-transferase 1 |
| | D86580 | 0.0731 | nuclear receptor subfamily 0, |
| | | | group B, member 2 |
| | BF550426 | 0.0391 | KDEL (Lys-Asp-Glu-Leu) endoplasmic |
| | | | reticulum protein retention |
| | | | receptor 2 |
| | AA818999 | 0.0296 | muscleblind-like 2 |
| | NM_019125 | 0.0167 | probasin |
| | AF150082 | −0.0141 | translocase of inner mitochondrial |
| | | | membrane 8 (yeast) homolog A |
| | BE118425 | −0.0781 | Arsenical pump-driving ATPase |
| | NM_017136 | −0.126 | squalene epoxidase |
| | Al171367 | −0.3222 | HSPC154 protein |
| | NM_019369 | −0.637 | inter alpha-trypsin inhibitor, |
| | | | heavy chain 4 |
| | Al137259 | −0.7962 | ESTs |
| SPLR | NM_017340 | 5.3688 | acyl-coA oxidase |
| | BF282712 | 4.1052 | ESTs |
| | NM_012489 | 3.8462 | acetyl-Co A acyltransferase 1 |
| | | | (peroxisomal 3-oxoacyl-Co A thiolase) |
| | BF387347 | 1.767 | ESTs |
| | K03249 | 1.7524 | enoyl-Co A, hydratase/3-hydroxyacyl |
| | | | Co A dehydrogenase |
| | NM_016986 | 0.0622 | acetyl-co A dehydrogenase, medium |
| | | | chain |
| | AB026291 | −0.7456 | acetoacetyl-CoA synthetase |
| | Al454943 | −1.6738 | likely ortholog of mouse porcupine |
| | | | homolog |

It is interesting to note that only three genes are common between these two linear classifiers, (K03249, BF282712, and BF387347) and even those are associated with different weights. While many of the genes may be different, some commonalities may nevertheless be discerned. For example, one of the negatively weighted genes in the SPLP derived signature is NM_{—}017136 encoding squalene epoxidase, a well-known cholesterol biosynthesis gene. Squalene epoxidase is not present in the SPLR derived classifier but aceto-acteylCoA synthetase, another cholesterol biosynthesis gene is present and is also negatively weighted.

Additional variant classifiers may be produced for the same classification task. For example, the average signature length (number of genes) produced by SPLP and SPLR, as well as the other algorithms, may be varied by use of the parameter p. Varying p can produce classifiers of different length with comparable test performance. Those classifiers are obviously different and often have no common genes between them (i.e. they do not overlap in terms of genes used).

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference. A practitioner of ordinary skill in the art may also find some helpful guidance by reviewing the attached appendix.

While the invention has been described with reference to the specific embodiments thereof, it should be understood by those skilled in the art that various changes may be made and equivalents may be substituted without departing from the true spirit and scope of the invention as defined by the claims. In addition, many modifications may be made to adapt a particular situation, material, composition of matter, method, process operation or operations, to the spirit and scope of the invention. All such modifications are intended to be within the scope of the claims. In particular, while the methods disclosed herein have been described with reference to particular operations performed in a particular order, it will be understood that these operations may be combined, sub-divided, or re-ordered to form an equivalent method without departing from the teachings of the invention. Accordingly, unless specifically indicated herein, the order and grouping of the operations is not a limitation of the invention.

What is claimed is:

1. A method of identifying a biological activity of a compound of interest, comprising:
providing a plurality of gene expression datasets associated with a first class of compounds having a first biological activity;
providing a plurality of gene expression datasets associated with a second class of compounds having a second biological activity;
deriving a linear classification rule based on said plurality of gene expression datasets; and
applying said linear classification rule to a set of gene expression levels associated with said compound of interest thereby determining whether said compound of interest has said first biological activity or said second biological activity.

2. The method of claim 1, wherein each dataset comprising a set of gene expression levels and a set of gene expression intervals.

3. The method of claim 1, wherein deriving said linear classification rule includes deriving a linear classification function.

4. The method of claim 3, wherein deriving said linear classification function includes reducing a value of a loss function associated with said plurality of gene expression datasets.

5. The method of claim 4, wherein reducing said value of said loss function includes reducing a worse-case value of said loss function.

6. The method of claim 3, wherein deriving said linear classification function includes identifying a set of classifiers that minimize a value of a loss function associated with said plurality of gene expression datasets.

7. The method of claim 6, wherein said loss function is associated with one of a support vector machine, logistic regression, and minimax probability machine.

8. A method of identifying a biological state of a biological sample, comprising:
providing a plurality of gene expression datasets, each gene expression dataset of said plurality of gene expression datasets including a set of gene expression levels and a set of gene expression intervals, said plurality of gene expression datasets including a first plurality of gene expression datasets associated with a first biological state and a second plurality of gene expression datasets associated with a second biological state;
deriving a linear classification rule based on said plurality of gene expression datasets; and
applying said linear classification rule to a set of gene expression levels associated with said biological sample to identify a biological state of said biological sample as one of said first biological state and said second biological state.

9. The method of claim 8, wherein said first biological state and said second biological state correspond to a normal condition and a disease condition, respectively.

10. The method of claim 8, wherein deriving said linear classification rule includes deriving a linear classification function.

11. The method of claim 10, wherein deriving said linear classification function includes reducing a value of a loss function associated with said plurality of gene expression datasets.

12. The method of claim 1, wherein reducing said value of said loss function includes reducing a worse-case value of said loss function.

13. The method of claim 10, wherein deriving said linear classification function includes identifying a set of classifiers that minimize a value of a loss function associated with said plurality of gene expression datasets.

14. The method of claim 13, wherein said loss function is associated with one of a support vector machine, logistic regression, and minimax probability machine.

15. A method for classifying a test gene expression dataset comprising:
providing a reference gene expression dataset;
deriving a linear classification rule by reducing the value of a loss function associated with said reference gene expression dataset; and
applying said linear classification rule to a test gene expression dataset thereby determining the classification of the test gene expression dataset.

16. The method of claim 15 wherein the reference gene expression dataset is a chemogenomic dataset based on in vivo compound treatments.

17. The method of claim 15 wherein the type of loss function is selected from the group consisting of support vector machine, logistic regression, and minimax probability machine.

18. A computer program product for classifying a test gene expression dataset comprising:
computer code for querying a reference gene expression dataset;
computer code for deriving a linear classification rule by reducing the value of a loss function associated with said reference gene expression dataset;
computer code for applying said linear classification rule to a test gene expression dataset and thereby determining the classification of the test gene expression dataset; and
computer code for outputting the test dataset classification to the user.

19. The computer code product of claim 18 wherein the type of loss function is selected from the group consisting of support vector machine, logistic regression, and minimax probability machine.
